(19) United States
(12) Patent Application Publication
(10) Pub. No.: US 2025/0049953 A1
(43) Pub. Date: Feb. 13, 2025

(54) TREATMENT AND METHOD FOR INHIBITING LATE NA CURRENT

(71) Applicant: THE TRUSTEES OF COLUMBIA UNIVERSITY IN THE CITY OF NEW YORK, New York, NY (US)

(72) Inventors: Nourdine CHAKOURI, New York, NY (US); Manu BEN JOHNY, New York, NY (US); Steven O. MARX, New York, NY (US)

(21) Appl. No.: 18/801,053

(22) Filed: Aug. 12, 2024

Related U.S. Application Data
(63) Continuation of application No. PCT/US2023/062512, filed on Feb. 13, 2023.
(60) Provisional application No. 63/309,049, filed on Feb. 11, 2022.

(51) Int. Cl. A61K 48/00; A61K 38/18; A61P 9/06; C12N 15/86

(52) U.S. Cl. CPC A61K 48/005; A61K 38/1825; A61P 9/06; C12N 15/86; C12N 2710/10343

(57) ABSTRACT

Treatments and methods for inhibiting late Na current use fibroblast growth factor homologous factor (FHF), an endogenous channel modulator, to inhibit late Na current with high potency. A minimal effector domain is engineered within FHF (the “FHF-inhibiting-X-region” (FixR)) as a peptide inhibitor of late Na current that may be delivered intracellularly, for example as a cell-penetrating peptide, or via viral or plasmid delivery. As a non-limiting example, human adenovirus type 5 may be genetically modified with the sequence 5′-ATGGCTGCGGCGATAGCCAGCTCCTTGATCCGGCAGAAGCGGCAGGCGAGGGAG TCCAACAGCGACCGAGTGTCGGCCTCCAAGCGCCGCTCCAGCCCCAGCAAAGAC GGGCGCTCC-3′ (SEQ ID NO: 1). As pathophysiological impact of late Na current extends beyond cardiac myocytes to other physiological settings, including neurons of the central and peripheral nervous system and skeletal muscle, these treatments and methods provide potential therapeutic avenues for a range of human ailments, including cardiac conditions, neurological/neuropsychiatric disorders, and skeletal muscle conditions. Neurological/neuropsychiatric disorders include, for example, epilepsy and autism spectrum disorders, pain-related diseases, and myotonia.

### CROSS-REFERENCE TO RELATED APPLICATION

This application is a continuation of International Application No. PCT/US2023/062512, filed Feb. 13, 2023, which claims benefit of U.S. Provisional Application No. 63/309,049 filed Feb. 11, 2022, the contents of which are hereby incorporated by reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 7,168 byte XML file named “44010-109US-PAT-CU22077_seq_listing.xml” created on Oct. 25, 2024.

### GOVERNMENT LICENSE RIGHTS

This invention was made with government support under grant no. NS110672, awarded by the National Institutes of Health (NIH). The government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### DISCUSSION

### 1. Technical Field

The disclosure of the present patent application relates to inhibiting late Na current, and particularly to treatment with a minimal effector domain engineered from within fibroblast growth factor homologous factor (FHF) as a peptide inhibitor of late Na current that may be delivered intracellularly, for example as a cell-penetrating peptide or via viral or plasmid delivery.

### 2. Background Art

According to the American Heart Association, cardiac arrhythmias are thought to cause 75% to 80% of cases of sudden cardiac death, which are estimated to result in 184,000 to 450,000 lives lost in the United States per year, leading to large social and economic burden. Dysfunction of the voltage-gated sodium channel NaV1.5, the predominant Na channel isoform in the heart, is a critical contributor to cardiac arrhythmia and other cardiac diseases. Typically, the ultra-rapid activation of NaV1.5 supports the fast upstroke and brisk spatial propagation of the cardiac action potential (AP), and the ensuing channel inactivation is essential for normal repolarization. Pathophysiologically, disruption of the inactivation process yields sustained depolarizing Na⁺ influx, called “late” or “persistent” Na current (I_{Na,L}), that thwarts normal repolarization and prolongs the AP. Consequently, increased late Na current is associated with multiple inherited and acquired diseases including long-QT syndrome 3 (LQTS3), dilated cardiomyopathy, heart failure and atrial fibrillation. As such, late Na current has emerged as a prominent drug target, and pharmacological agents that selectively prevent late Na current without affecting the peak current are highly sought after.

Multiple cellular and molecular mechanisms have been identified to upregulate late Na current, including (i) gain-of-function channelopathic mutations that disrupt the channel inactivation machinery, (2) NaV1.5 phosphorylation that is upregulated in cardiac pathologies such as heart failure, (3) various ischemic metabolites, (4) reactive oxygen species, (5) channel interacting proteins, and (6) other post-translational mechanisms. This staggering diversity in pathophysiological mechanisms poses a key challenge in developing effective approaches to curtail late Na current.

To date, the predominant strategy for inhibition late sodium current has been small molecules, such as Ranolazine, GS-458967, GS-462808, and F15845. These compounds typically target the transmembrane local anesthetic binding site and exhibit variable efficacy and selectivity in inhibiting late versus peak Na current. Although these compounds have demonstrated some therapeutic potential, they have significant limitations. Specifically, Ranolazine, the most well-established late Na current inhibitor, has suboptimal potency and has non-selectivity interactions with other ionic currents, including Ca²⁺ channels and K⁺ channels. By comparison, GS-458967 has high efficacy for blocking late Na current, however, it has high brain penetration and use-dependent block for many neuronal Na channels, resulting in potential central nervous system side effects. GS-462808 caused liver lesions during the acute animal toxicity tests. F15845 also blocked late sodium current with a very high efficacy; however, the last experimental reports about F15845 were published in 2010, where it prevented ischemia induced arrhythmias in rats. Therefore, identifying orthogonal strategies for late sodium current inhibition are critical for developing new therapeutics.

Interestingly, in native cardiomyocytes, the actual magnitude and functional manifestation of I_{Na,L} changes is often variable, suggesting that there may be endogenous factors that prevent late Na current. A prime example of such incongruity stems from our prior analysis of calmodulin (CaM) regulation of Na_{V}1.5. In heterologous cells, mutations that weaken CaM binding yield a consistent increase in I_{Na,L}; yet, ablating CaM binding to Na_{V}1.5 paradoxically showed no such change in murine ventricular cardiomyocytes. This cellular variability in the I_{Na,L} may be clinically relevant, as many Na_{V}1.5 channelopathies exhibit variable expressivity whereby mutations have overlapping disease phenotypes. Factors that contribute to this variability are largely unknown, although vital to understanding disease mechanisms. Furthermore, such endogenous factors that prevent I_{Na,L} could inform on new approaches and channel interfaces that could be leveraged for developing therapeutics. To this end, we recently found that the well-established Na_{V}1.5 modulator, intracellular fibroblast grown factor (FGF) homologous factors (FHF or iFGF) can inhibit I_{Na,L}. Yet, the precise contribution of FHF in tuning I_{Na,L} in cardiac physiology and pathophysiology remains to be fully determined, and furthermore, the full potential of this likely powerful modulatory scheme for engineering selective I_{Na,L} inhibitors is unrealized. Importantly, as increased I_{Na,L} is associated with neuronal Na channelopathies linked to epilepsy as well as in skeletal muscle linked to paramyotonia congenita and periodic paralysis, in depth mechanistic analysis of cardiac Na_{V}1.5 may have broader ramifications.

Thus, a treatment and method for inhibiting late Na current solving the aforementioned problems is desired.

### SUMMARY DISCLOSURE

The treatment and method for inhibiting late Na current uses fibroblast growth factor homologous factor (FHF), an endogenous channel modulator, to inhibit late Na current with high potency. A minimal effector domain is engineered within FHF (referred to as “FHF-inhibiting-X-region” (FixR)) as a peptide inhibitor of late Na current that may be delivered intracellularly, for example as a cell-penetrating peptide or via viral or plasmid delivery. As a non-limiting example of one method of delivery to a patient, human adenovirus type 5 may be genetically modified with the sequence 5′-ATGGCTGCGGCGATAGCCAGCTCCTTGA TCCGGCAGAAGCGGCAGGCGAGGGAGTCCAACAGCGACCGAGTGTCGGCCTCCA AGCGCCGCTCCAGCCCCAGCAAAGACGGGCGCTCC-3′ (SEQ ID NO: 1). This sequence is one sequence coding for the FixR peptide, in this instance with the amino acid sequence MAAAIASSLIRQKRQARESNSDRVSASKRRSSPSKDGRS (SEQ ID NO: 4). The FixR peptide sequence includes at least about 35 amino acids, which may be modified or extended while retaining efficacy.

Other examples of methods of delivery to a patient may use, for example, a viral vector that is not based on human adenovirus type 5, a plasmid vector, or any other suitable delivery vector or mechanism known to the practitioner at that time. As pathophysiological impact of late Na current extends beyond cardiac myocytes to other physiological settings, including neurons of the central and peripheral nervous system and skeletal muscle, the present treatment and method provides a potential therapeutic avenue for a wide range of human ailments, including neurological/neuropsychiatric disorders, such as epilepsy and autism spectrum disorders, pain-related diseases, and myotonia.

We reasoned that endogenous Na channel modulators that tune Na channel inactivation may provide a new avenue for developing selective late Na current blockers. To this this end, the family of fibroblast growth factor homologous factors (FHF) are attractive candidates as they are cytosolic proteins that interact with the NaV carboxy-terminus and as they are well-established to tune channel inactivation. Recent findings suggest that FHFs are also potent inhibitors of late Na current. Four distinct genes encode FHF1-4 isoforms that show tissue-specific expression. Furthermore, alternative splicing of each isoform generates additional variants encoding different amino-terminal sequences. Our systematic analysis revealed that distinct FHF isoforms/splice-variants exhibit varying degrees of late Na current inhibition, with some variants demonstrating ˜20-100-fold reduction in late Na current. Importantly, further analysis demonstrates that FHF1A, a variant that is not natively expressed in the human heart, is the most potent inhibitor of late Na current.

In view of the above, we undertook extensive mechanistic analysis to determine minimal structural domains in FHF that permit inhibition of late Na current. Our analysis identified a minimal 39-amino-acid segment of FHF1A amino-terminus that confers strong inhibition late sodium current when expressed as a peptide. Further deletions, including a previously identified long-term inactivation particle, yielded sharply-diminished or no inhibition of late Na current. The present treatment and method corresponds to this minimal domain (FixR), its derivatives, and potential use as either a genetically-encoded or peptidic late Na current blocker for experimental or therapeutic purposes.

As FixR is a peptide that acts on intracellular channel domains, its potential application requires intracellular delivery of FixR. We developed two strategies for intracellular delivery of FixR into cardiomyocytes or into other physiological settings: viral delivery of FixR into adult cardiomyocytes and other excitable cells; and intracellular delivery facilitated by cell penetrating peptides. With regard to viral delivery of FixR into adult cardiomyocytes and other excitable cells, robust intracellular expression of FixR may be attained through adenoviral, lentiviral, or AAV transduction. To demonstrate this possibility, we generated adenovirus (Ad-FixR) that expresses FixR tagged to venus fluorescent protein as a reporter. This strategy also permits cell-type specific expression by leveraging specific promoters or by using AAV with different serotypes that may allow targeted delivery of FixR. We confirmed intracellular expression of FixR in iPSC-derived cardiomyocytes and in cultured myocytes 1-2 days following infection of both cell types with Ad-FixR.

With regard to intracellular delivery facilitated by cell penetrating peptides, in order to engineer FixR as a peptide-inhibitor of late sodium current a cell-penetrating moiety was attached. A bevy of cell penetrating peptides have been previously developed for intracellular delivery, including those that target specific cell types. Here, we fused FixR with a protein transduction domain from HIV-1 trans-activators of transcription (TAT), which facilitates entry into the cells (termed FixR-CPP). One embodiment of the FixR-CPP construct has the protein sequence YGRKKRRQRRRAAAIASSLIRQKRQARESNSERVSASKRRSSPSKG (SEQ ID NO: 2), wherein the FixR portion has the sequence AAAIASSLIRQKRQARESNSER VSASKRRSSPSKG (SEQ ID NO: 5), and the CPP portion has the sequence YGRKKRRQRRR (SEQ ID NO: 6).

To monitor its uptake, we synthesized FixR-CPP, whose amino-terminus is tagged with FITC, such that cellular entry may be quantified by monitoring FITC fluorescence intensity. Our analysis with HEK293 cells showed robust uptake of FixR-CPP at 10 μM concentration following 2 hours of incubation, with ˜90% of cells showing increased fluorescence compared to background. Incubation of freshly dissociated adult ventricular cardiomyocytes also confirmed robust entry with a 10 μM concentration and 2 hours of incubation. Furthermore, electrophysiological analysis demonstrates the exquisite capability of FixR to inhibit I_{Na,L} in adult ventrical cardiomyocytes.

These and other features of the present subject matter will become readily apparent upon further review of the following specification.

### BRIEF DESCRIPTION OF THE DRAWINGS

FIG. 1A shows epifluorescence images of cultured aMVMs from IQ/AA^{tg} mouse transduced with FHF2 shRNA.

FIG. 1B shows exemplar multichannel Na_{V} recordings from pseudoWT^{tg} (pWT^{tg}) mice, showing minimal late channel openings in the late phase (gray shaded region); n=11 cells, where the inset shows an enlarged view of the late phase.

FIG. 1C shows exemplar multichannel NaV recordings from aMVMs from IQ/AA^{tg} mice, showing show minimal late Na channel openings (n=12 cells).

FIG. 1D and FIG. 1E show exemplar multichannel NaV recordings from the application of FHF2 shRNA, revealing an increase in late channel openings for cultured aMVMs from IQ/AA^{tg} (FIG. 1E; n=11 cells), but not pWT^{tg} mice (FIG. 1D; n=10 cells).

FIG. 1F and FIG. 1G show exemplar multichannel NaV recordings from scrambled shRNA, revealing no change in I_{Na,L} (n=11-13 cells).

FIG. 1H is a bar graph showing the quantification and population data of changes in I_{Na,L} upon manipulating FHF2 levels in non-transgenic (non-TG), pWT^{tg}, and IQ/AA^{tg} aMVMs. Rₚₑᵣₛᵢₛₜ is the average open probability (P_{O}) in the late phase normalized by the peak P_{O}. Each bar and error are mean±s.e.m. Statistical analysis was performed using the Kruskal-Wallis test followed by Dunn's multiple comparisons test, showing **p=0.0072 between FHF2 shRNA and scrambled shRNA for IQ/AA^{tg} (n=3-4 mice).

FIG. 2A shows exemplar multichannel NaV recordings showing that wild-type Na_{V}1.5 exhibits minimal I_{Na,L} when heterologously expressed in HEK293 cells (n=13 cells).

FIG. 2B, 2C and 2D show exemplar multichannel NaV recordings showing that the co-expression of FHF1_{A} (FIG. 2B; n=7 cells), FHF1_{B} (FIG. 2C; n=11 cells), and FHF2_{S} (FIG. 2D; n=10 cells), showing a yield of minimal change in I_{Na,L}.

FIG. 2E shows exemplar multichannel NaV recordings showing that Na_{V}1.5 IQ/AA mutant abolishing interaction of CaM, and showing elevated I_{Na,L} (n=13 cells).

FIG. 2F, FIG. 2G and FIG. 2H show exemplar multichannel NaV recordings showing that the co-expression of either FHF1_{A} (FIG. 2F; n=11 cells) or FHF2_{S} (FIG. 2H; n=12 cells) inhibited I_{Na,L}, suggesting that these variants are “protective”. However, FHF1_{B} yielded an ˜3-fold increase in I_{Na,L} (FIG. 2G; n=13 cells) and is considered “non-protective”.

FIG. 2I shows exemplar multichannel NaV recordings showing that that the LQTS3-linked ΔKPQ mutant demonstrates increased I_{Na,L} at baseline (n=12 cells).

FIG. 2J, FIG. 2K and FIG. 2L show exemplar multichannel NaV recordings showing that that both the FHF1_{A} (FIG. 2J; n=12 cells) and FHF2_{S} (FIG. 2L; n=9 cells), but not FHF1_{B} (FIG. 2K; n=6 cells), show a strong reduction in I_{Na,L}.

FIG. 2M is a bar graph, summarizing changes in I_{Na,L} quantified as Rₚₑᵣₛᵢₛₜ upon co-expression of various FHF isoforms/splice-variants. Each bar and error are mean±s.e.m. Statistical analysis was performed by the Kruskal-Wallis test, followed by Dunn's multiple comparisons test: ***p<0.001; **p<0.01; and *p<0.05 for each mutant compared to no FHF. §§p<0.01 compared to wild-type Na_{V}1.5 at baseline.

FIG. 2N schematically illustrates a competitive scheme of I_{Na,L} regulation by “protective” versus “non-protective” FHF variants.

FIG. 2O is a graph showing that the transfection of varying DNA concentrations of FHF1_{B} versus FHF2_{S} yields an increasing relationship between I_{Na,L} and the ratio of cDNA transfected (ρ) (n=5-12 cells). The inset shows exemplar traces.

FIG. 2P is a plot showing the ToR-Ord in silico model showing AP prolongation and emergence of AP alternans for IQ/AA mutant depending on the relative expression of protective versus non-protective FHF isoform.

FIG. 2Q and FIG. 2R are graphs showing that the APD of IQ/AA mutant increases as a function ρ at both 45 bpm (FIG. 2Q) and 80 bpm (FIG. 2R) pacing. No change in APD is observed with wild-type.

FIG. 3A schematically shows that channelopathic mutations in disparate channel domains and channel phosphorylation upregulate I_{Na,L}.

FIG. 3B shows exemplar multichannel NaV recordings showing that FHF1A inhibits I_{Na,L} for LQTS3-linked nonsense mutation E[1810]Δ that disrupts the canonical FHF-binding CTD interface (n=6 cells).

FIG. 3C is a bar graph summary of I_{Na,L} for various channel mutations linked to LQTS3, atrial fibrillation, and mixed-syndrome phenotype, showing that FHF1_{A} significantly reduces I_{Na,L} for all mutations. INa,L of each mutant is compared in the presence versus absence of FHF1A (n=7-15 cells) ***p<0.001; **p<0.01; and *p<0.05 by the Mann-Whitney U-test.

FIG. 3D shows exemplar multichannel NaV recordings showing that FHF1ₐ inhibits I_{Na,L} of wild-type Na_{V}1.5 upregulated by co-expression of PKA catalytic subunit (PKA_{cat}) (n=13 cells).

FIG. 3E is a bar graph summarizing the effect of FHF in inhibiting phosphorylation-dependent I_{Na,L} triggered by PKA_{cat} or constitutively active CaMKII (CaMKII_{T286D}) (n=10-12 cells), where the dashed line indicates I_{Na,L} for wild-type Na_{V}1.5 absent kinase. Each bar and error represents mean±s.e.m. Statistical analysis was performed by the Kruskal-Wallis test followed by Dunn's multiple comparisons test: ***p<0.001; **p<0.01; and *p<0.05 compared to no FHF for a given kinase. $p<0.05 compared to wild-type Na_{V}1.5 at baseline.

FIG. 3F shows exemplar multichannel NaV recordings showing that transient PKA activation by 5 μM forskolin results in minimal I_{Na,L} increase for cultured non-TG aMVMs transduced with scrambled shRNA (n=5 cells) (top), and that the same maneuver results in a marked increase in I_{Na,L} when FHF2 is downregulated by shRNA (n=9 cells) (bottom).

FIG. 3G is a bar graph showing forskolin-dependent changes in Rₚₑᵣₛᵢₛₜ of Na channels in non-TG aMVM with scrambled versus FHF2 shRNA. Each bar and error represents mean±s.e.m. *p=0.018 by the Mann Whitney U-test.

FIG. 3H schematically shows potential mechanisms underlying FHF regulation of I_{Na,L}.

FIG. 3I and FIG. 3J are plots showing single channel recordings of IFM/IQM mutant that disrupts the inactivation particle shows persistent openings at baseline (FIG. 2I; n=7 cells) and robust inhibition in the presence of FHF1_{A} (FIG. 2J; n=13 cells). In each figure, the top shows exemplar traces, and the bottom shows the ensemble average.

FIG. 3K and FIG. 3L are plots showing that FHF1A reduces the likelihood of late channel openings for LILA/WICW mutation that allosterically obstructs the transmembrane receptor site for IFM motif (n=7-10 cells).

FIG. 4A is a multichannel recording showing minimal I_{Na,L} in iPSC-CMs derived from healthy donors (n=11 cells).

FIG. 4B is a multichannel recording showing LQTS3 patient-derived iPSC-CM with a heterozygous Na_{V}1.5 ΔKPQ mutation show increased I_{Na,L} (n=11 cells).

FIG. 4C and FIG. 4D are multichannel recordings showing that adenoviral expression of FHF2 shRNA resulted in no appreciable change in I_{Na,L} for both iPSC-CM lines (p=0.10 by Kruskal-Wallis test, n=9-15 cells).

FIG. 4E and FIG. 4F are multichannel recordings showing transduction of scrambled shRNA also yielded no change in I_{Na,L} (n=9-13 cells).

FIG. 4G is a multichannel recording showing over-expression of FHF1_{A} yielded no change in I_{Na,L} for iPSC-CMs from healthy donors (n=12 cells).

FIG. 4H is a multichannel recording showing that FHF1_{A} diminishes I_{Na,L} in LQTS3 (ΔKPQ) iPSC-CMs (n=13 cells).

FIG. 4I is a bar graph summary of Rₚₑᵣₛᵢₛₜ, confirming strong inhibition of pathogenic I_{Na,L} by FHF1_{A} in LQTS3 (ΔKPQ) iPSC-CMs. Each bar and error represents mean±s.e.m. Statistical analysis was performed by Kruskal-Wallis followed by Dunn's multiple comparisons test **p=0.0012.

FIG. 5A shows exemplar multichannel NaV recordings showing that FHF1_{A} amino terminus suffices to inhibit I_{Na,L} of Na_{V}1.5 IQ/AA mutant (n=9 cells).

FIG. 5B shows exemplar multichannel NaV recordings showing that further truncation of the amino terminus to residues 1-39 also reduced I_{Na,L} akin to full-length FHF1_{A} (n=10 cells).

FIG. 5C is a bar graph summarizing Rₚₑᵣₛᵢₛₜ for Na_{V}1.5 IQ/AA mutant co-expressed with various FHF1A amino-terminal truncations compared to Ranolazine (n=6-11 cells). FixR is identified, being composed of FHF1A amino-terminal residues 1-39, as a minimal domain sufficient for strong I_{Na,L} inhibition. Statistical analysis was performed by one-way ANOVA followed by Dunnett's multiple comparisons test: ***p<0.001; **p<0.01; and *p<0.05.

FIG. 5D and FIG. 5E show exemplar multichannel NaV recordings showing that FixR inhibits I_{Na,L} of both Na_{V}1.5 ΔKPQ (FIG. 5D; n=11 cells) and E1784K mutation (FIG. 5E; n=9 cells).

FIG. 5F and FIG. 5G show exemplar multichannel NaV recordings showing that FixR reduces I_{Na,L} of Na_{V}1.5 trigged by co-expression of PKA_{cat} (n=9 cells) and CaMKII_{T286D} (n=9 cells).

FIG. 5H is a bar graph demonstrating FixR as a strong and generalizable inhibitor for both channelopathic and phosphorylation-dependent I_{Na,L} (n=9-13 cells). Each bar and error is mean±s.e.m. ***p<0.001; **p<0.01; and *p<0.05 by the Mann Whitney U-test.

FIG. 5I shows exemplar multichannel NaV recordings showing that I_{Na,L} of LQTS3 (ΔKPQ) iPSC-CMs remains elevated upon expression of GFP (n=11 cells).

FIG. 5J shows exemplar multichannel NaV recordings showing that adenoviral expression of FixR reduced I_{Na,L} for LQTS3 (ΔKPQ) iPSC-CMs (n=13 cells).

FIG. 5K is a bar graph demonstrating the effect of FixR to inhibit pathogenic I_{Na,L} of LQTS3 (ΔKPQ) iPSC-CMs. ***p<0.001 by the Mann Whitney U-test.

FIG. 6A schematically shows design of FixR as a cell permeable peptide (FixR-cpp).

FIG. 6B is a histogram obtained from flow cytometric analysis showing increased FITC fluorescence in single HEK293 cells following 2-hour incubation with FixR-cpp at various doses.

FIG. 6C is a plot showing a dose-dependent increase in FixR-cpp uptake into HEK293 cells.

FIG. 6D shows epifluorescence images showing uptake of FixR into freshly-dissociated aMVM.

FIG. 6E and FIG. 6F show that flow cytometric analysis shows dose-dependence uptake of FixR in aMVM, with FIG. 6E showing a histogram of single-myocyte FITC fluorescence.

FIG. 6G shows exemplar multichannel NaV recordings showing that acute application of FixR-cpp to HEK293 cells transfected with Na_{V}1.5 ΔKPQ mutant reduces I_{Na,L} (n=6 cells). Baseline measurement is shown on top, with the bottom following FixR-cpp.

FIG. 6H is a bar graph confirming inhibition of I_{Na,L} by FixR-cpp (n=6-18 cells). Each bar and error is mean±s.e.m. The black dots are Rₚₑᵣₛᵢₛₜ measurements obtained prior to addition of FixR-cpp, the blue dots are Rₚₑᵣₛᵢₛₜ measurements from cells in the same dish following 2-hour incubation with FixR-cpp, and the light gray dots are pooled data reproduced from FIG. 2M to facilitate comparison. Statistical analysis: *p=0.048, unpaired t-test comparing Rₚₑᵣₛᵢₛₜ for cell on the same dish before and after addition of FixR-cpp.

FIG. 6I shows exemplar multichannel NaV recordings showing that ShRNA suppression of FHF2 results in increased I_{Na,L} in cultured aMVM obtained from IQ/AA^{tg} mice as in FIG. 1E (n=16 cells) (top). The bottom shows that acute application of FixR-cpp reduces I_{Na,L} (n=10 cells).

FIG. 6J is shows population data confirming strong inhibition of I_{Na,L} following 2 hour incubation with FixR-cpp. Statistical analysis: ***p<0.001 by Mann Whitney U-test.

FIG. 7A-FIG. 7D|shRNA suppression of FHF2 has no effect on I_{Na,L} in non-TG aMVM. a, Epifluorescence images of cultured aMVMs from non-TG mice transduced with FHF2 shRNA. b, Exemplar multichannel Na_{V} recordings from uninfected non-TG mice show minimal late channel openings (n=9 cells). Format as in FIG. 1b. c-d, Application of both FHF2shRNA and scrambled shRNA revealed no change in I_{Na,L} openings for cultured aMVMs from non-TG mice (n=10-11 cells). Population data are shown in FIG. 1.

FIG. 8A schematically shows various FHF2 splice variants generated by alternate start sites. Conserved exons that encode the core domain are shown in red boxes labeled E2 to E5.

FIG. 8B, FIG. 8C, FIG. 8D and FIG. 8E show exemplar recordings of LQTS3-linked ΔKPQ mutant heterologously expressed in HEK293 cells in the presence of various FHF2 splice variants (format as in FIG. 2A; n=9-12 cells).

FIG. 8F, FIG. 8G, FIG. 8H and FIG. 8I are exemplar recordings of IQ/AA mutant in the presence of FHF2 splice variants (format as in FIG. 2A; n=9-15 cells).

FIG. 8J is a bar graph summarizing changes in I_{Na,L} quantified as Rₚₑᵣₛᵢₛₜ upon co-expression of FHF splice variants. Each bar and error represents mean±s.e.m. Statistical analysis was performed by the Kruskal-Wallis test followed by Dunn's Multiple comparisons test, ***p<0.001; **p<0.01; and *p<0.05 when compared to no FHF control of each mutant channel. FHF2_{S}, and FHF2_{VY} yielded a partial reduction in I_{Na,L} for both the IQ/AA and the ΔKPQ mutant, while FHF2_{V} and FHF2_{U} resulted in no change.

FIG. 9A shows exemplar multichannel recordings from recombinant Na_{V}1.5 wild type in HEK293 cells showing minimal late channel openings in the presence of FHF3_{A} (n=13 cells) (format as in FIG. 1B; population data is shown in FIG. 2M).

FIG. 9B shows exemplar recordings showing a partial reduction in late channel openings for Na_{V}1.5 IQ/AA mutant channel in the presence of FHF3_{A} (n=15 cells) (population data is shown in FIG. 2M).

FIG. 10A schematically illustrates the α-subunit of Na_{V}1.5 channel showing missense (grey) and nonsense (red) mutations in disparate channel domains.

FIG. 10B shows exemplar multichannel recordings of Na_{V}1.5 F1759A in the absence (top) and presence of FHF1_{A} (bottom).

FIG. 10C, FIG. 10D, FIG. 10E and FIG. 10F show exemplar recordings suggesting that FHF1_{A} inhibits E1784K, S1904L, Q1909R, and S[1885]* mutant.

FIG. 11A schematically shows Na_{V}1.5 phosphorylation by PKA and CaMKII upregulates I_{Na,L}.

FIG. 11B shows exemplar multichannel recordings from wild-type Na_{V}1.5 upregulated by co-expression of constitutively active CaMKII (CaMKII_{T286D}) (n=10 cells).

FIG. 11C and FIG. 11D show exemplar multichannel recordings showing that both FHF1_{A} (FIG. 11C; n=9 cells) and FHF2_{S} (FIG. 11D; n=9 cells) inhibits CaMKII_{T286D}-triggered I_{Na,L} of wild-type Na_{V}1.5.

FIG. 11E shows exemplar multichannel recordings showing that FHF2_{S} inhibits PKA-dependent I_{Na,L} of wild-type Na_{V}1.5 (n=7 cells, similar to the effects of FHF1_{A} in FIG. 3D).

FIG. 12A shows FL distributions for Na_{V}1.5 IFM/IQM at baseline (black line and gray shaded area) and upon overexpression of FHF1A (red line and rose shaded area). FL denotes the probability that the first opening occurred at time<t. FHF1_{A} had minimal effect on the FL distribution for the IFM/IQM mutant.

FIG. 12B shows FL distributions for Na_{V}1.5 LILA/WICW at baseline (gray shaded area) and under overexpression of FHF1A (rose shaded area). FHF1_{A} decreased the pedestal value of FL for the LILA/WICW mutant suggesting that FHF1A increases closed-state inactivation. p<0.001 by KS-test.

FIG. 12C shows an open-duration (OD) distribution showing Na_{V}1.5 IFM/IQM tallies the durations of a single sojourn to the open state. FHF1_{A} shortens the OD distribution for the IFM/IQM mutant, hinting at potential increase in the rate constant for inactivation from the open state. p<0.001 by KS-test.

FIG. 12D shows an OD distribution, showing FHF1A has no effect on OD distribution of the LILA/WICW mutant.

FIG. 13A shows the sequence alignment of various FHF1A amino-terminal peptides utilized to identify a minimal effector domain.

FIG. 13B schematically illustrates potential interaction between the peptides and Na_{V}1.5 channel.

FIG. 13C, FIG. 13D, FIG. 13E and FIG. 13F show exemplar multichannel recordings from Na_{V}1.5 IQ/AA mutant showing an increase late channel openings when co-expressed with 1-18 (FIG. 13C; n=9 cells), 18-39 (FIG. 13D; n=7 cells), 12-17 (FIG. 13E; n=6 cells), or 9-32 (FIG. 13F; n=9 cells) peptides.

FIG. 13G shows exemplar multichannel recordings showing that ranolazine yields a modest inhibition of I_{Na,L} for Na_{V}1.5 IQ/AA mutant (n=11 cells).

FIG. 13H and FIG. 13I show exemplar multichannel recordings showing that both FixR (FIG. 13H; n=13 cells) and ranolazine (FIG. 13I; n=10 cells) inhibits I_{Na,L} for Na_{V}1.5 S1904L mutant channel.

FIG. 13J is a bar graph showing Rₚₑᵣₛᵢₛₜ for Na_{V}1.5 S1904L mutant channel in the presence of FixR or ranolazine. The black dashed line is I_{Na,L} for Na_{V}1.5 S1904L at baseline, and the blue dashed line is I_{Na,L} for Na_{V}1.5 S1904L co-expressed with FixR. Each bar and error is mean±s.e.m. Statistical analysis was performed by one-way ANOVA followed by Dunnet's multiple-comparisons test ***p<0.001.

FIG. 13K shows exemplar multichannel recordings showing that FixR also strongly inhibits I_{Na,L} for Na_{V}1.5 Δ1885 mutant channel (n=11 cells).

FIG. 13L and FIG. 13M show exemplar multichannel recordings showing adenoviral expression of both GFP (FIG. 13L; n=9 cells) and FixR (FIG. 13M; n=10 cells) yielded no change in I_{Na,L} in iPSC-CMs derived from healthy donors (HD).

FIG. 14A shows representative flow cytometric data showing the percentage of cellular uptake of FixR (FITC positive) into HEK293 cells and effect on cytotoxicity determined using a far red (APC) DEAD cell stain (Invitrogen). Cells are incubated with varying concentrations of FixR-cpp for 2 hours. Appreciable cellular uptake is observed between 5 and 25 μM. Minimal cell death is observed in the presence of up to 10 μM FixR-cpp.

FIG. 14B shows epifluorescence images showing FixR-cpp uptake into freshly dissociated cardiomyocytes from IQ/AA^{tg} mice (n=3 mice).

FIG. 15A shows representative multichannel recordings of wild-type Na_{V}1.1 channel, important for neuronal function.

FIG. 15B shows that FixR has minimal effect on wild-type Na_{V}1.1 channel.

FIG. 15C shows increased I_{Na,L} observed with the Na_{V}1.1 H1929Q mutation linked to Early infantile epileptic encephalopathy. This mutation increases late Na current.

FIG. 15D shows that FixR strongly reduced I_{Na,L} for the Na_{V}1.1 H1929Q mutation.

FIG. 15E shows bar graph summary of FixR effects on Na_{V}1.1.

FIG. 16A shows representative multichannel recordings of wild-type Na_{V}1.4 in the presence (bottom) and absence (top) of FHF1_{A}, a protective FHF variant.

FIG. 16B shows that Na_{V}1.4 channelopathic mutation linked to cold-aggravated myotonia results in an increase in I_{Na,L}. FHF1_{A} co-expression strongly reduces I_{Na,L}.

FIG. 16C. Bar graph summarizes effect of FHF1_{A} on both wild-type and mutant Na_{V}1.4 channels.

Similar reference characters denote corresponding features consistently throughout the attached drawings.

### DETAILED DESCRIPTION OF THE EMBODIMENTS

The treatment and method for inhibiting late Na current uses fibroblast growth factor homologous factor (FHF), an endogenous channel modulator, to inhibit late Na current with high potency. A minimal effector domain is engineered within FHF (referred to as “FHF-inhibiting-X-region” (FixR)) as a peptide inhibitor of late Na current that may be delivered intracellularly, such as, for example, as a cell-penetrating peptide or via viral delivery. Other means for delivery include, for example, use of a viral vector, or use of a plasmid vector. As a non-limiting example of one method of delivery to a patient using one example of a viral vector, human adenovirus type 5 may be genetically modified with the sequence 5′-ATGGCTGCGGCGATAGCCAGCTCCTTGATCCGGCAGAAGCGGCAGGCGAGGGAG TCCAACAGCGACCGAGTGTCGGCCTCCAAGCGCCGCTCCAGCCCCAGCAAAGAC GGGCGCTCC-3′ (SEQ ID NO: 1)-one embodiment of a DNA sequence encoding for FixR. The FixR peptide includes a minimum of about 35 amino acids. In one embodiment, for example, the FixR peptide comprises AAAIASSLIRQKRQARESNSERVSASKRRSSPSKG (SEQ ID NO: 5). In another embodiment, the FixR peptide comprises MAAAIASSLIRQKRQAR ESNSDRVSASKRRSSPSKDGRS (SEQ ID NO: 4). As will be apparent to one of skill in the art, some degree of modifications to these exemplary FixR peptide sequences may be successfully employed.

As pathophysiological impact of late Na current extends beyond cardiac myocytes to other physiological settings, including neurons of the central and peripheral nervous system and skeletal muscle, the present treatment and method provides a potential therapeutic avenue for a wide range of human ailments, including neurological/neuropsychiatric disorders, such as epilepsy and autism spectrum disorders, pain-related diseases, and myotonia.

In one embodiment, a treatment composition for inhibiting late Na current comprises an mRNA encoding the FixR peptide. The treatment composition may be delivered using a viral vector, or a plasmid vector, or any other suitable vector or mechanism., including without limitation delivery by nanoparticles, such as, for example, lipid nanoparticles.

In another embodiment, the treatment composition comprises a viral or plasmid vector genetically modified with a coding sequence of the FixR peptide. The coding sequence may comprise, for example, the sequence 5′-ATGGCTGCGGCGATAG CCAGCTCCTTGATCCGGCAGAAGCGGCAGGCGAGGGAGTCCAACAGCGACCGAG TGTCGGCCTCCAAGCGCCGCTCCAGCCCCAGCAAAGACGGGCGCTCC-3 (SEQ ID NO: 1). The viral or plasmid vector may comprise, for example, human adenovirus type 5.

In another embodiment, the treatment composition comprises a peptide inhibitor of late Na current, wherein the peptide inhibitor is FixR. The FixR is optionally fused to a cell penetrating peptide (FixR-CPP).

In another embodiment, the treatment composition comprises a cell penetrating peptide. The FixR-CPP fusion peptide may, for example, include the protein sequence YGRKKRRQRRRAAAIASSLIRQKRQARESNSERVSASKRRSSPSKG (SEQ ID NO: 2), including both a FixR portion (AAAIASSLIRQKRQARESNSERVSASKRRSSPSKG (SEQ ID NO: 5)) and the CPP portion (YGRKKRRQRRR (SEQ ID NO: 6)) of the fused peptide.

In another embodiment, the treatment composition is useful for treating one or more of the following: a cardiac pathology, a neurological/neuropsychiatric disorder, and a skeletal muscle condition. The cardiac pathology may comprise arrhythmia.

Another embodiment provides a method for inhibiting late Na current, comprising administering to a patient an effective amount of any of these treatment compositions.

In another embodiment, the method for inhibiting late Na current in a patient comprises administering to the patient a treatment composition comprising an effective amount of human adenovirus type 5 genetically modified with the sequence 5′-ATGGCTGCGGCGATAGCC AGCTCCTTGATCCGGCAGAAGCGGCAGGCGAGGGAGTCCAACAGCGACCGAGTG TCGGCCTCCAAGCGCCGCTCCAGCCCCAGCAAAGACGGGCGCTCC-3 (SEQ ID NO: 1).

In another embodiment, the method for inhibiting late Na current in a patient comprises administering to the patient a treatment composition comprising an effective amount of FixR-CPP, with the combined protein sequence YGRKKRRQRRRAAAIASSLIRQKRQAR ESNSERVSASKRRSSPSKG (SEQ ID NO: 2).

In any of these methods, the patient may be treated, for example, for one or more of the following: a cardiac pathology, a neurological/neuropsychiatric disorder, and a skeletal muscle condition. The patient may be treated, for example, for one or more of the following: arrhythmia, epilepsy and autism spectrum disorders, pain-related diseases, and myotonia.

### Discussion

We reasoned that endogenous Na channel modulators that tune Na channel inactivation may provide a new avenue for developing selective late Na current blockers. To this this end, the family of fibroblast growth factor homologous factors (FHF) are attractive candidates as they are cytosolic proteins that interact with the NaV carboxy-terminus and as they are well-established to tune channel inactivation. Recent findings suggest that FHFs are also potent inhibitors of late Na current. Four distinct genes encode FHF1-4 isoforms that show tissue-specific expression. Furthermore, alternative splicing of each isoform generates additional variants encoding different amino-terminal sequences. Our systematic analysis revealed that distinct FHF isoforms/splice-variants exhibit varying degrees of late Na current inhibition, with some variants demonstrating ˜20-100-fold reduction in late Na current, while others evoked no change. Importantly, further analysis demonstrates that FHF1A, a variant that is not natively expressed in the human heart, is the most potent inhibitor of late Na current.

In view of the above, we undertook extensive mechanistic analysis of FHF1/2 to determine minimal structural domains that permit inhibition of late Na current. Our analysis identified a minimal 39-amino-acid segment of FHA1A/2 amino-terminus that confers strong inhibition late sodium current when expressed as a peptide. Further deletions, including a previously identified long-term inactivation particle, yielded sharply diminished or no inhibition of late Na current. The present treatment and method correspond to this minimal domain (FixR), its derivatives, and potential use as either a genetically encoded or peptidic late Na current blocker for experimental or therapeutic purposes.

As FixR is a peptide that acts on intracellular channel domains, its potential application requires intracellular delivery of FixR. We developed two strategies for intracellular delivery of FixR into cardiomyocytes or into other physiological settings: viral delivery of FixR into cardiomyocytes and other excitable cells; and intracellular delivery facilitated by cell penetrating peptides. With regard to viral delivery of FixR into cardiomyocytes and other excitable cells, robust intracellular expression of FixR may be attained through adenoviral, lentiviral, or AAV transduction. To demonstrate this possibility, we generated adenovirus (Ad-FixR) that expresses FixR tagged to venus fluorescent protein as a reporter. This strategy also permits cell-type specific expression by leveraging specific promoters or by using AAV with different serotypes that may allow targeted delivery of FixR. We confirmed intracellular expression of FixR in iPSC-derived cardiomyocytes and in cultured myocytes 1-2 days following infection of both cell types with Ad-FixR.

With regard to intracellular delivery facilitated by cell penetrating peptides, in order to engineer FixR as a peptide-inhibitor of late sodium current a cell-penetrating moiety was attached. A bevy of cell penetrating peptides have been previously developed for intracellular delivery, including those that target specific cell types. Here, we fused FixR with a protein transduction domain from HIV-1 trans-activators of transcription (TAT), which facilitates entry into the cells (termed FixR-CPP). The FixR-CPP peptide has, for example, the combined protein sequence YGRKKRRQRRRAAAIASSLIRQKRQARESNSERVSASKRRSSPSKG (SEQ ID NO: 2). To monitor its uptake, we synthesized FixR-CPP, whose amino-terminus is tagged with FITC, such that cellular entry may be quantified by monitoring FITC fluorescence intensity. Our analysis with HEK293 cells showed robust uptake of FixR-CPP at 10 μM concentration following 2 hours of incubation, with ˜90% of cells showing increased fluorescence compared to background. Incubation of freshly dissociated ventricular cardiomyocytes also confirmed robust entry with a 10 μM concentration and 2 hours of incubation. Furthermore, electrophysiological analysis demonstrates the exquisite capability of FixR to inhibit I_{Na,L} in adult ventrical cardiomyocytes.

Here, we sought to determine how FHFs orchestrate cardiac Na_{V}1.5 regulation, and whether this mechanism can be leveraged to devise novel strategies to inhibit pathogenic I_{Na,L}. Synergistic experiments in HEK293 cells, cardiomyocytes differentiated from induced pluripotent stem cells (iPSC-CMs), and cultured adult mouse ventricular cardiomyocytes (aMVM), revealed that FHF regulation of I_{Na,L} is isoform-specific, whereby select isoforms/variants inhibit I_{Na,L} (“protective” variants), while other variants have either no effect or may exacerbate I_{Na,L} (“non-protective” variants). We hypothesize that the balance between endogenously expressed “protective” and “non-protective” FHF variants in cardiomyocytes may ultimately dictate the arrhythmogenic phenotype. Armed with these insights, we engineered the above minimal domain derived from “protective” FHFs as a selective and potent inhibitor of pathogenic I_{Na,L}. Overall, these findings lend new insights into the molecular mechanism for FHF regulation of I_{Na,L} and potential pathophysiological changes that ultimately lead to cardiac instability.

### Endogenous FHF2 Tunes I_{Na,L} of Mutant Na_{V}1.5 in Murine Cardiomyocytes

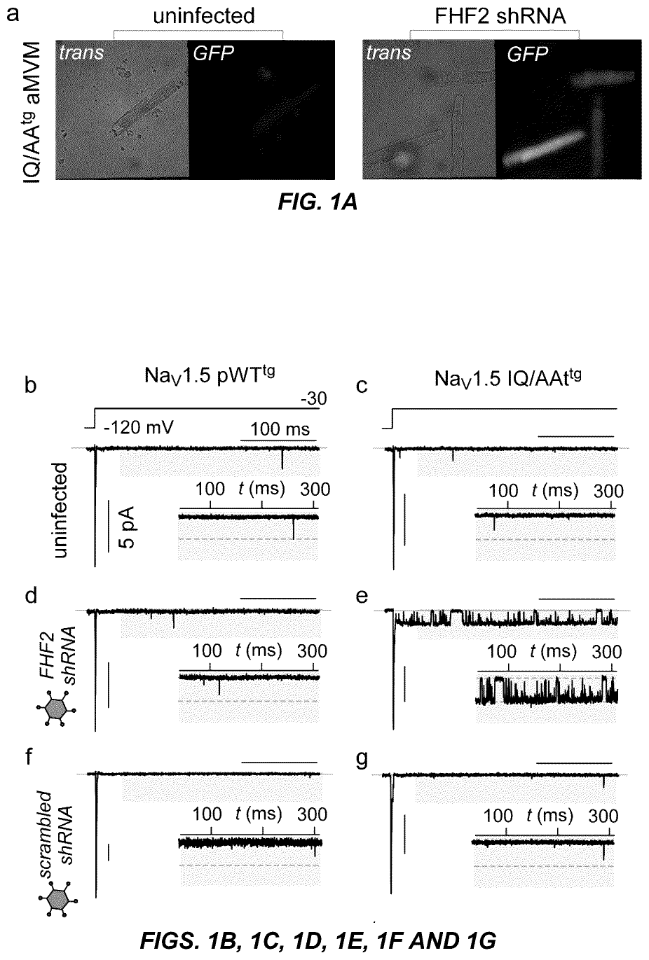

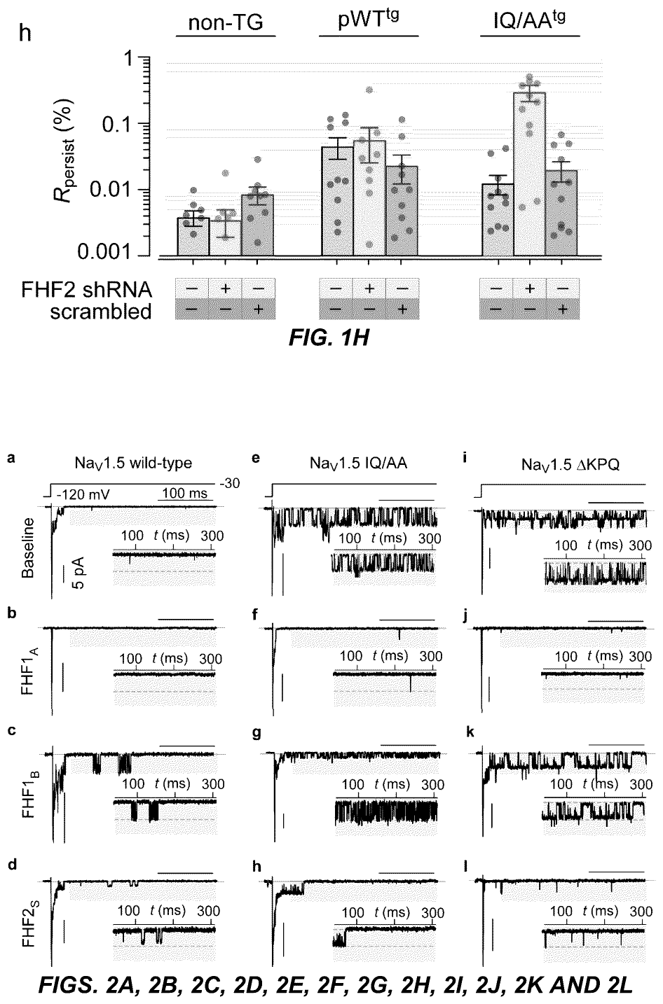

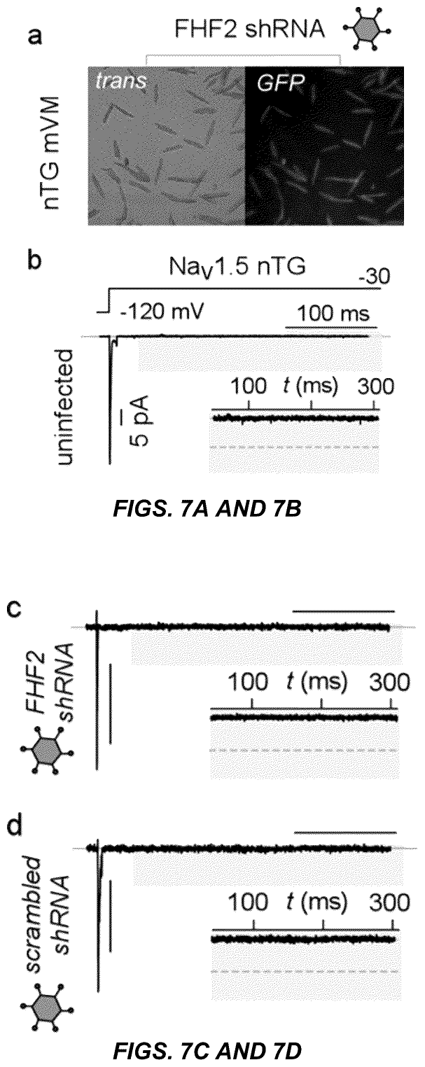

As FHF2 is natively expressed in the murine heart, we considered whether manipulation of ambient FHF levels in adult mouse ventricular myocytes (aMVM) tunes I_{Na,L}. To do so, we utilized our recently published transgenic mouse models expressing (1) pseudo-wild-type Na_{V}1.5 (pWT), and (2) IQ/AA mutant channels that abolish the interaction of CaM, a de facto channel subunit involved in the modulation of late Na current. When recombinantly expressed, IQ/AA mutant channels exhibit elevated I_{Na,L}. However, our previous studies have shown that this mutation has surprisingly little effect on I_{Na,L} in murine myocytes. Whether this discrepancy stems from I_{Na,L} modulation by endogenous FHF in cardiomyocytes is not fully established. Accordingly, we undertook cell-attached multichannel recordings to measure changes in the magnitude of I_{Na,L} upon downregulating FHF2 levels using shRNA. Cultured myocytes from both pWT and IQ/AA mice (FIG. 1A) exhibited minimal I_{Na,L} at baseline, similar to freshly dissociated myocytes in our previous studies (FIGS. 1B, IC and 1H). Of note, the amplitude of I_{Na,L} here is quantified as Rₚₑᵣₛᵢₛₜ, the ratio of the average open probability (P_{O}) following 50 ms of depolarization and the peak P_{O}. This approach allows robust quantification of the likelihood of late channel openings, while still maintaining the cytosolic milieu and intact signaling. Downregulation of FHF2 by adenovirally transduced shRNA had no effect on I_{Na,L} of pWT mice (FIGS. 1D and 1H), as expected given the strong inactivation of wild-type Na_{V}1.5. The same maneuver, however, revealed a marked increase in I_{Na,L} of aMVMs from IQ/AA mutant mice (FIGS. 1E and 1H). Expression of scrambled shRNA had no effect on I_{Na,L} in aMVMs from both pWT and IQ/AA mutant mice (FIGS. IF-1H). Similar to pWT, I_{Na,L} of non-transgenic mice was also insensitive to FHF2 manipulation (FIG. 1H and 7A-7D). These results demonstrate that FHF2 is an endogenous inhibitor of I_{Na,L}, that may counteract increased late Na current due to Na_{V}1.5 mutations.

### FHF Modulation of Late Na Current is Isoform/Splice-Variant Specific

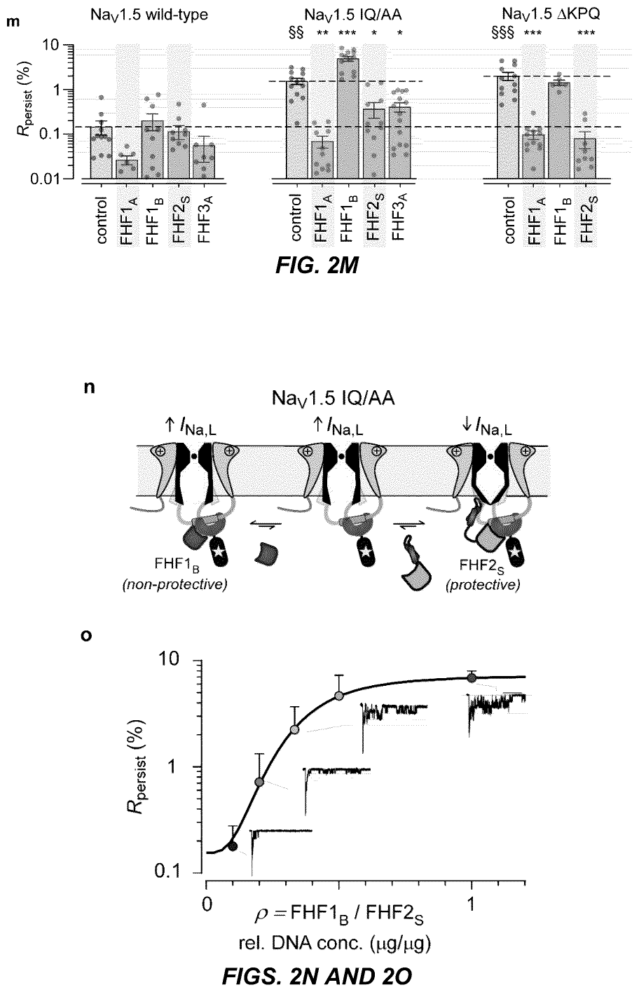

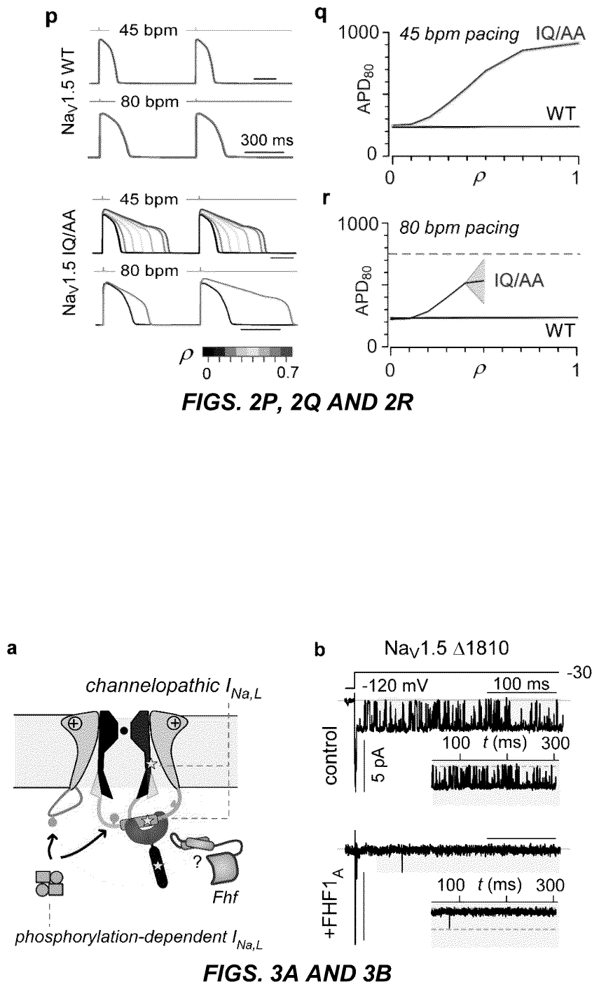

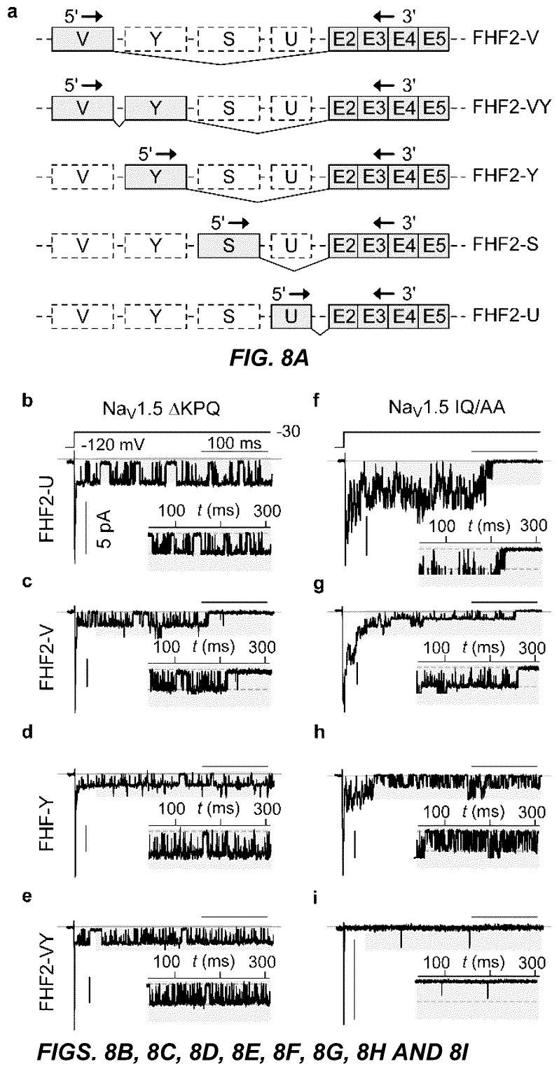

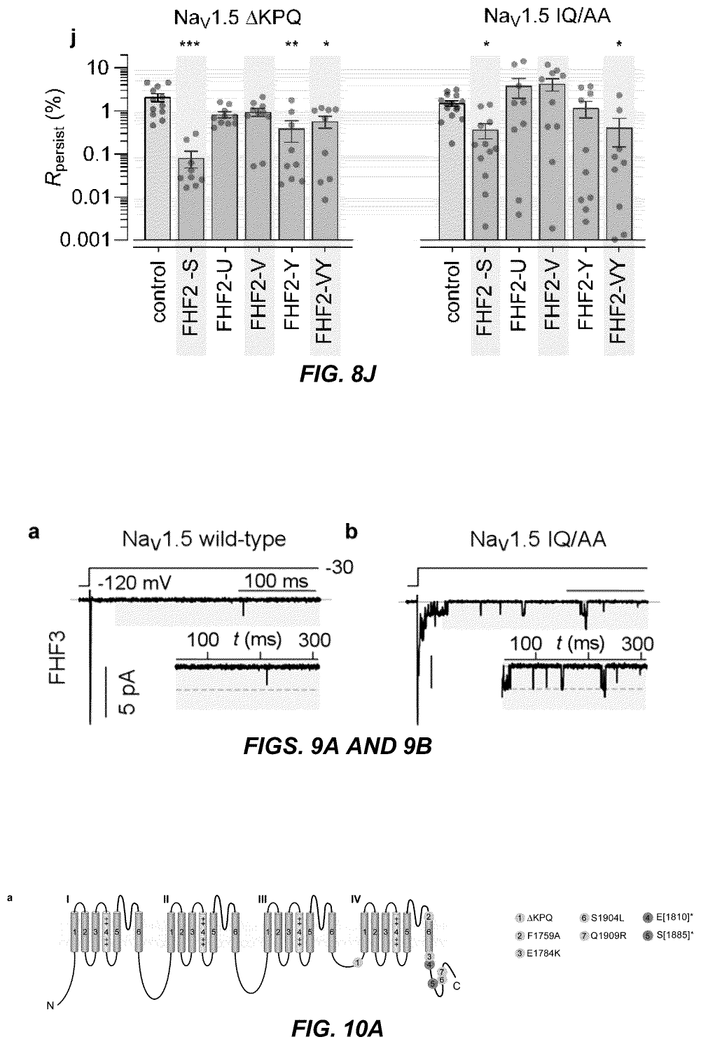

Intracellular FHFs are encoded by four distinct genes (FHF1/iFGF12, FHF2/iFGF13, FHF3/iFGF11, FHF4/iFGF14) that are differentially expressed in various excitable cells. Adding further complexity, all four FHF isoforms undergo alternative splicing that generate variants with distinct amino termini. In the murine heart, select FHF2 splice-variants are predominant, while in the human heart FHF1_{B} is the dominant isoform, although certain FHF2 splice variants are also expressed. Given this diversity, we sought to determine whether FHF regulation of I_{Na,L} is specific for certain variants by comparing the effect of multiple FHFs on I_{Na,L} triggered by structure guided and pathogenic Na_{V}1.5 mutants. As HEK293 cells have minimal endogenous FHFs, this system serves as a convenient platform to dissect mechanisms. Consistent with previous studies, wild-type Na_{V}1.5 exhibits minimal I_{Na,L} (FIGS. 2A and 2M). Furthermore, FHF isoforms evoked no further change in I_{Na,L} of wild-type channels (FIGS. 2B-2D and 2M). By comparison, both the IQ/AA mutation (FIGS. 2E and 2M), designed to mimic channelopathic mutations in the Na_{V}1.5 CT, and the ΔKPQ mutation, linked to LQTS3, demonstrated elevated I_{Na,L} (FIGS. 21 and 2M). Co-expression of FHF1_{A} resulted in a strong reduction in I_{Na,L} for both mutations (FIGS. 2F, 2J and 2M), reminiscent of the protective nature of endogenous FHF2 in mouse cardiomyocytes. By comparison, co-expression of the FHF1_{B} variant did not yield a similar reduction in I_{Na,L}; instead, we observed an ˜4-fold increase in I_{Na,L} for the IQ/AA mutation (FIGS. 2G and 2M), and no appreciable change for the ΔKPQ mutation (FIGS. 2K and 2M). These results suggest that FHF regulation of I_{Na,L} is splice-variant specific, with some variants serving a protective role by inhibiting I_{Na,L}, while others are inert or may exacerbate I_{Na,L}. Thus informed, we dissected the effect of FHF2-3 isoforms/splice-variants on I_{Na,L}. We found that FHF2_{S} (FIGS. 2H, 2L and 2M), FHF2_{VY}, FHF2_{Y}, and FHF3_{A} variably reduced I_{Na,L} for IQ/AA and ΔKPQ mutants (FIGS. 8A-8J, 9A and 9B), and are therefore termed “protective” variants. By contrast, FHF2_{U} and FHF2_{V} yielded minimal change in I_{Na,L} (FIGS. 2M and 8A-8J), and are therefore classified as “non-protective” variants.

The divergence in functional effects of FHF splice-variants/isoforms suggests that the net magnitude of I_{Na,L} for a given mutation may be tuned by the relative cytosolic concentration of “protective” versus “non-protective” FHF variants (FIG. 2N). To test this possibility, we co-expressed cDNAs of FHF1_{B}, a “non-protective” variant, along with FHF2_{S}, a “protective” variant at varying ratios and quantified changes in I_{Na,L} for the Na_{V}1.5 IQ/AA mutant. Multichannel recordings revealed an increasing Hill-Langmuir relationship for Rₚₑᵣₛᵢₛₜ when plotted against the ratio of FHG1_{B} to FHF2_{S} cDNA (ρ), with the half-maximal effect observed at ρ˜0.4, i.e. with ˜2.5 fold higher expression of FHF2_{S} compared to FHF1_{B} (FIG. 2O). Having identified this empirical relationship, we considered the impact of FHF regulation of I_{Na,L} on cardiac AP morphology. We modified the late Na current component of the ToR-ORd in silico human ventricular AP model to be dependent on the relative concentrations of FHF1_{B} and FHF2_{S}. As expected, simulations with wild-type Na_{V}1.5 showed no change in action potential duration (APD) as a function of ρ (FIGS. 2P, 2Q and 2R). Simulations incorporating the Na_{V}1.5 IQ/AA mutant, however, showed AP prolongation (at low heart rates) and AP alternans (at high heart rates) with increasing ρ, consistent with increased I_{Na,L} and arrhythmogenicity (FIGS. 2P, 2Q and 2R). In all, these results suggest that the relative expression of “protective” versus “non-protective” FHF variants may constitute a continuously tunable rheostat for I_{Na,L} and APD in cardiomyocytes.

### Generality of I_{Na,L} Inhibition by “Protective” FHF Variants

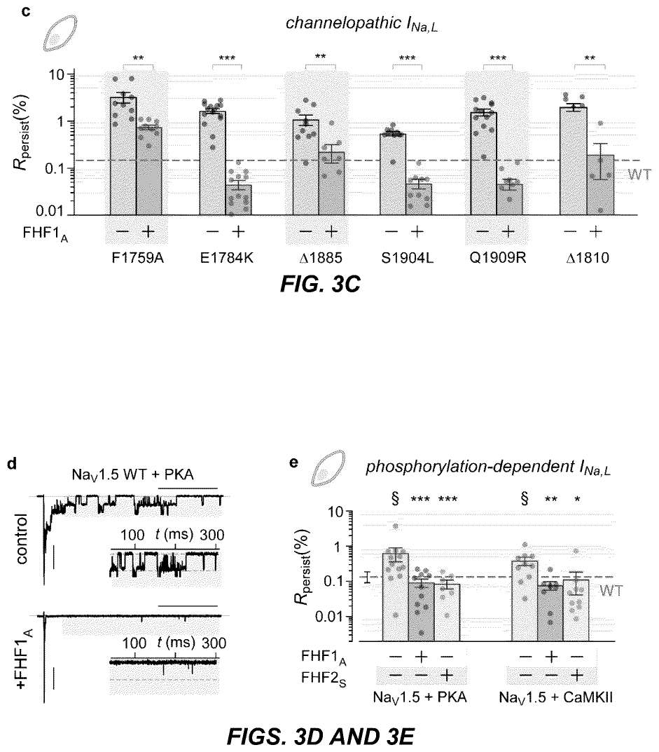

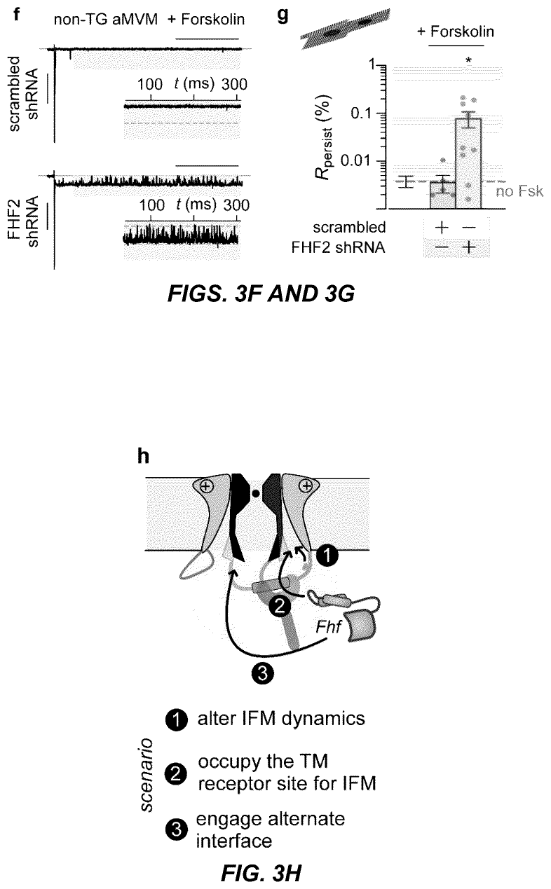

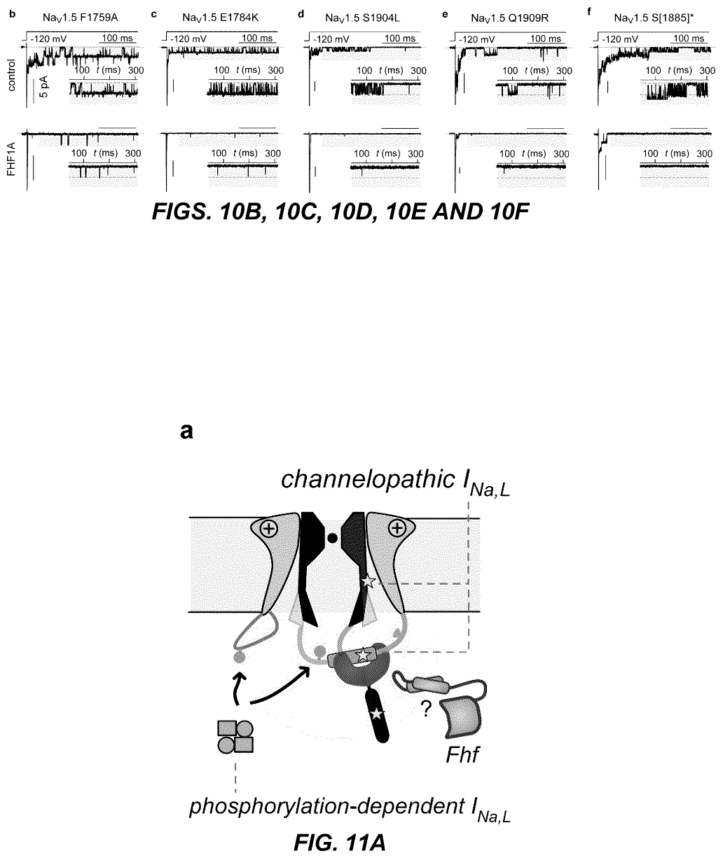

Pathological I_{Na,L} is triggered by diverse mechanisms including (i) channelopathic mutations in disparate structural domains and (ii) post-translational modifications that include channel phosphorylation (FIG. 3A). Our analysis of Na_{V}1.5 IQ/AA and ΔKPQ mutants suggests that “protective” FHF variants confer strong inhibition of I_{Na,L}. Yet, the generality of this modulation is unknown, although critical for dissecting pathophysiological relevance. We thus probed whether I_{Na,L} of various disease-linked mutations are inhibited by FHF1_{A}, the most potent I_{Na,L} inhibitor for both IQ/AA and ΔKPQ mutant channels. We first considered E1784K, S1904L, and Q1909R (FIG. 10A) in the channel carboxy-tail (CT) linked to a mixed-syndrome phenotype (i.e., both LQTS3 and Brugada Syndrome). We found that all three mutations increased I_{Na,L}, while co-expression of FHF1_{A} reversed this increase to wild-type levels (FIGS. 3C and 10C-10E). In like manner, F1759A, linked to increased I_{Na,L} and atrial fibrillation in mice, also upregulated I_{Na,L}. FHF1_{A} partially reversed this increase (FIGS. 3C and 10A-10F). Finally, we considered two nonsense mutations (E[1810]* and S[1885]*) in the channel CT that disrupts the canonical FHF-interacting interface. Of note, the E[1810]* truncation ablates the FHF binding interface in the Na_{V}1.5 CT. In both cases, we observed a baseline increase in I_{Na,L} (FIGS. 3B, 3C and 10F). Surprisingly, FHF1_{A} strongly diminished I_{Na,L} for both mutants (FIGS. 3B, 3C and 10F), suggesting that FHF interaction with the Na_{V}1.5 CT is not necessary for functional regulation of I_{Na,L}.

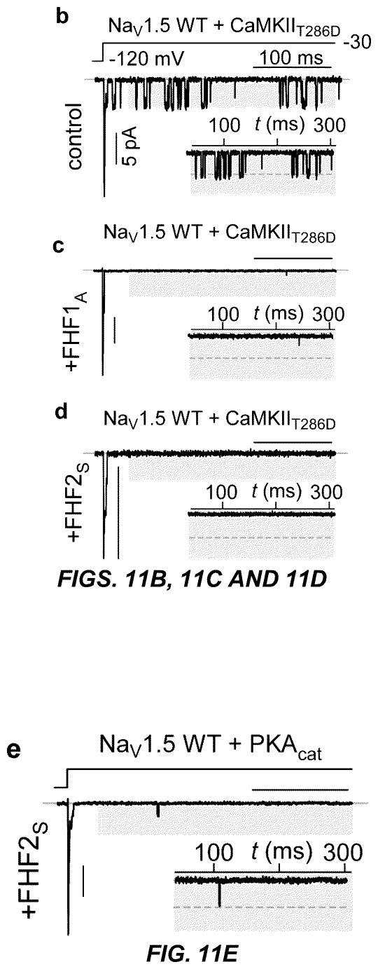

Beyond channelopathic mutations, I_{Na,L} is also upregulated by phosphorylation of Na_{V}1.5, a critical factor for arrhythmogenesis in heart failure and myocardial ischemia. As such, we sought to determine whether “protective” FHF variants may inhibit phosphorylation-dependent I_{Na,L}. To maximally upregulate I_{Na,L}, we co-expressed wild-type Na_{V}1.5 with either (1) the catalytic C_{α} subunit of protein kinase A (PKA) or (2) a constitutively-active Ca²⁺/CaM dependent kinase II (CaMKII_{T286D}) mutant in HEK293 cells. This maneuver likely triggers runaway phosphorylation of Na_{V}1.5 and thereby furnishes a baseline to discern any potential FHF-dependent change. As expected, we observed an ˜5 fold increase in I_{Na,L} in the presence of the PKA C_{α} subunit (FIGS. 3D and 3E), and a 3-fold increase with CaMKII_{T286D} (FIGS. 3E and 11B). Co-expression of either FHF1_{A} or FHF2_{S} sufficed to largely reverse this increase (FIGS. 3D, 3E and 11C-11E). Encouraged by these findings, we dissected the functional role of this modulation in native cardiomyocytes (FIGS. 3F and 3G). At baseline, application of 5 μM Forskolin did not appreciably change I_{Na,L} in cultured aMVM from non-transgenic mice (FIGS. 3F and 3G). However, following shRNA-mediated silencing of FHF2, we found a striking 20-fold increase in I_{Na,L} (FIGS. 3F and 3G), suggesting that endogenous FHF2 plays a vital role in tuning phosphorylation-dependent I_{Na,L}. Furthermore, these findings establish the generality, potency, and physiological relevance of FHF regulation of I_{Na,L} triggered by various mechanisms.

As such, we sought to examine biophysical mechanisms underlying I_{Na,L} inhibition by “protective” FHF variants/isoforms. In particular, Na_{V} inactivation is driven by an allosteric obstruction of the channel pore by the isoleucine-phenylalanine-methionine (IFM) motif in the channel III-IV linker, while I_{Na,L} results from a disruption of this process. “protective” FHFs may inhibit I_{Na,L} via three broad schemes: FHFs may (i) enhance translocation of the ‘IFM’ motif to its receptor site, (ii) mimic the ‘IFM’ motif to interact with the receptor site, or (iii) act elsewhere on the channel (FIG. 3H). To distinguish between these possible mechanisms, we disabled fast inactivation of Na_{V}1.5 by either substituting the central phenylalanine with glutaminc (IFM/IQM), or by a triple mutation in domain I S6 (LILA/WICW) that allosterically occludes the ‘IFM’ receptor interface. Single channel recordings of IFM/IQM mutant showed burst-like openings with strongly disrupted inactivation, as in our previous study (FIG. 3I). Co-expression of FHF1_{A}, however, increased inactivation as evident from exemplar traces and the ensemble average relationship (FIG. 3J), thus excluding possibility i.

In a like manner, recordings of LILA/WICW mutant revealed flickery openings and a strong reduction in inactivation (FIG. 3K). Co-expression of FHF1_{A} revealed enhanced inactivation (FIG. 31), an outcome inconsistent with possibility ii. These findings, combined

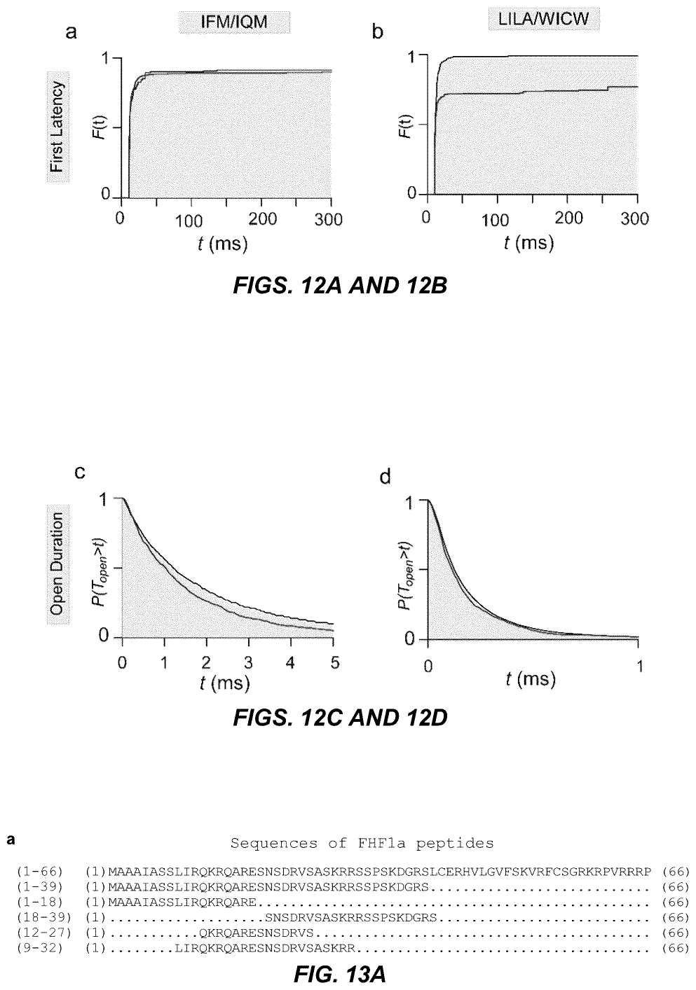

with the ability of FHF to inhibit I_{Na,L} of E[1810]* mutant that abolishes the canonical CT binding interface suggests that FHF acts on an as-yet-unidentified interface on the Na_{V} channel to trigger an orthogonal inactivation mechanism. For both inactivation-deficient mutants, the excess inactivation followed a single-exponential decay with an approximately similar time constant (17.8 ms for IFM/IQM and 12.3 ms for LILA/WICW mutation). To garner further insights, we tallied first latencies (FL, the time elapsed before the first opening) and open durations (OD) in the presence and absence of FHF1_{A} for both mutant channels. For the IFM/IQM mutant, we observed that FHF1_{A} diminishes the OD distribution without an appreciable effect on the FL distribution, suggesting that FHF enhances inactivation from the open state (FIGS. 12A and 12C). However, for the LILA/WICW mutant, we found that FHF1_{A} had minimal effect on the OD distribution, however, it decreased the pedestal value of the FL distribution, suggesting that FHF1A enhanced closed-state inactivation for this mutation (FIGS. 12B and 12D). This switch in the microscopic inactivation properties is evocative of earlier models of Na channel inactivation proposed by Aldrich-Corey-Stevens.

### “Protective” FHF Inhibits I_{Na,L} in LQTS3 iPSC-CM

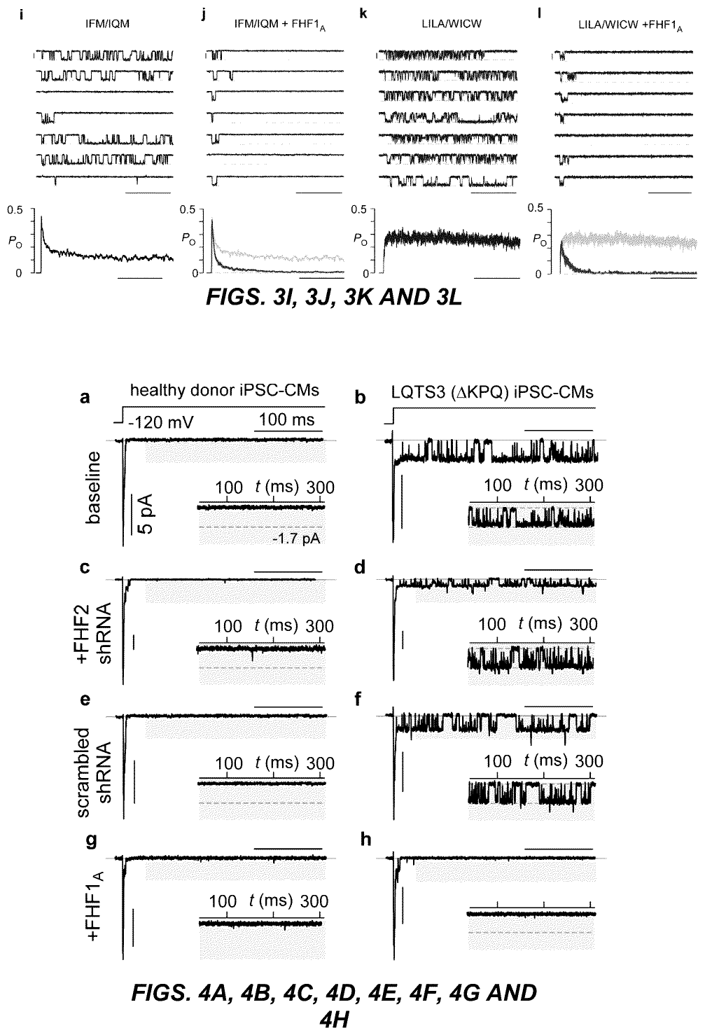

To dissect the potential pathophysiological relevance of I_{Na,L} regulation by FHF, we studied cardiomyocytes (CMS) differentiated from either LQTS3 patient-derived iPSCs bearing the heterozygous ΔKPQ mutation in the SCN5A gene (LQTS3 (ΔKPQ) line) or those from healthy donors (HD line). Following 30-day differentiation of CMs, robust spontaneous contraction of myocytes was observed. We undertook cell-attached multichannel recordings to quantify I_{Na,L} in both HD and LQTS3 (ΔKPQ) iPSC-CM lines. Unlike the HD line (FIGS. 4A and 4I), the LQTS3 (ΔKPQ) line showed appreciable late channel openings, consistent with elevated baseline I_{Na,L} (Rₚₑᵣₛᵢₛₜ˜0.73%; FIGS. 4B and 4I). As FHF2-shRNA targets both murine and human sequence, we probed changes in I_{Na,L} following transduction of FHF2-shRNA or scrambled shRNA. Both maneuvers resulted in no change in I_{Na,L} in either HD or LQTS3 (ΔKPQ) lines (FIGS. 4C-4F and 4I). Adenoviral transduction of FHF1_{A} also revealed no change in I_{Na,L} for the HD line (FIGS. 4G and 4I). In contrast, overexpression of the most potent “protective” FHF isoform, FHF1_{A}, resulted in an inhibition of I_{Na,L} for the LQTS3 (ΔKPQ) line (FIGS. 4H and 4I). These findings illustrate the exquisite capability of “protective” FHF variants to inhibit pathogenic I_{Na,L}. As increased I_{Na,L} is a commonality for many Na channelopathies, manipulating FHF modulation of Na_{V}1.5 may furnish a new avenue to counter this pathophysiological change.

### FixR—a Minimal FHF Domain that Inhibits I_{Na,L}

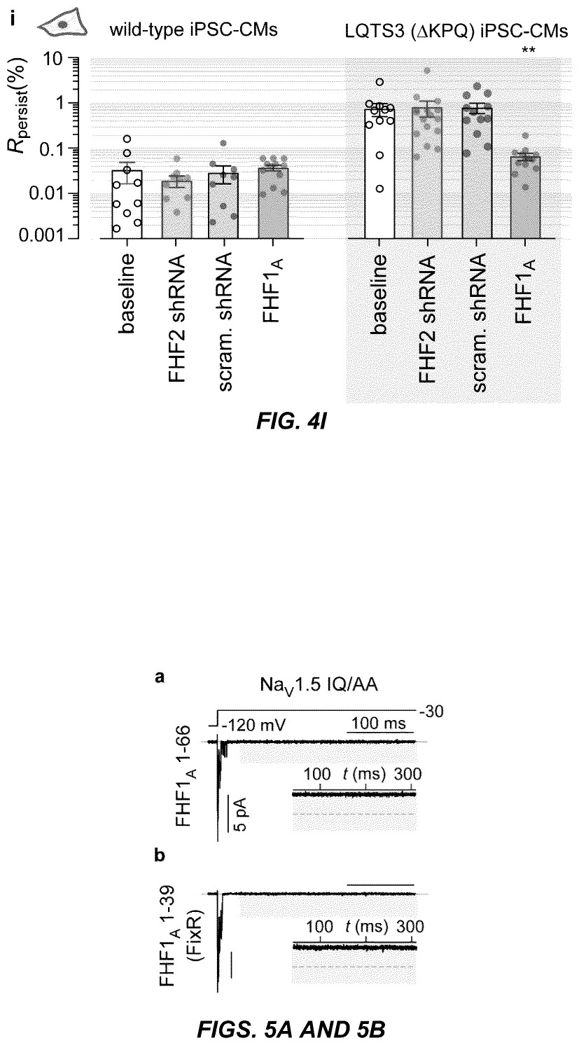

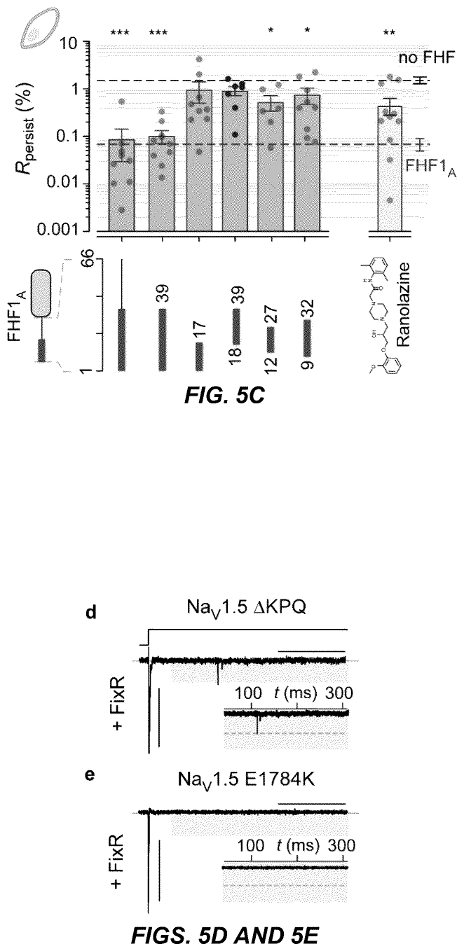

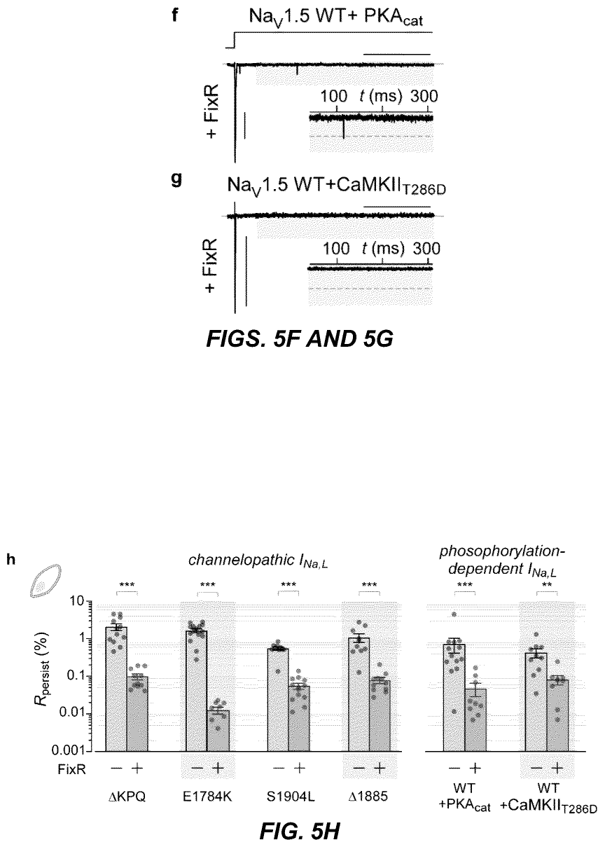

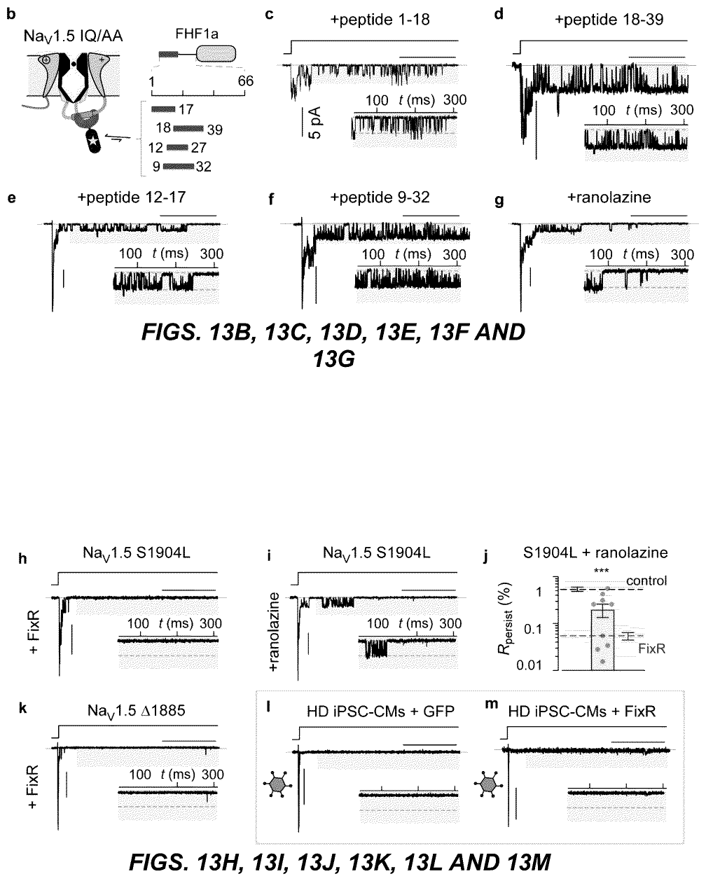

The efficacy and generality of FHF1_{A} in diminishing pathogenic I_{Na,L} suggests that this isoform may provide an ideal substrate for developing new mechanism-inspired selective inhibitors of late Na current. To do so, we sought to localize a minimal FHF segment that mediates I_{Na,L} inhibition. Sequence comparison of the “protective” FHF1_{A} versus the “non-protective” FHF1_{B} suggested that the key domains responsible for functional I_{Na,L} regulation resides in the divergent amino-terminal segment (FIG. 13A). Indeed, expression of the entire FHF1_{A} amino terminus as a peptide strongly inhibited I_{Na,L} of Na_{V}1.5 IQ/AA mutant channels, akin to full length FHF1_{A} (FIGS. 5A and 5C). To further localize key effector domains, we generated multiple truncated versions of this region and found that the amino-terminal 39 residues sufficed for strong inhibition of I_{Na,L} (FIGS. 5B and 5C). Further truncations diminished efficacy of I_{Na,L} inhibition (FIGS. 5C and 13B-13F). This suggests that the 39-amino acid segment constitutes a minimal effector domain for I_{Na,L} inhibition. As such, we termed this segment the FHF inhibiting x Region (FixR). Thus apprised, we probed the efficacy of FixR in inhibiting I_{Na,L} resulting from various disease linked Na_{V}1.5 channelopathic mutations and channel phosphorylation. Indeed, FixR evoked an ˜10-fold inhibition of I_{Na,L} in nearly all cases (FIGS. 5D-5H and 13H-13K). Importantly, ranolazine is a well-established I_{Na,L} inhibitor with a higher efficacy in blocking I_{Na,L} as compared to I_{Na,peak}. Indeed, we observed that incubation with 10 μM ranolazine resulted in a modest 3-fold reduction in I_{Na,L} for both IQ/AA and S1904L mutant (FIGS. 5C, 13G, 13I and 13J). By comparison, FixR evoked a pronounced 10- to 15-fold reduction in I_{Na,L} (FIG. 5C), thus highlighting the potency of FixR for I_{Na,L} inhibition as compared to currently existing gold-standard late Na current inhibitor.

To ascertain the functional relevance of FixR in tuning pathogenic I_{Na,L}, we studied its effect in LQTS3 (ΔKPQ) iPSC-CMs. To do so, we generated adenovirus that bicistronically expresses FixR and venus fluorescent protein using a P2A self-cleaving peptide. Transduction of FixR diminished I_{Na,L} in the LQTS3 (ΔKPQ) iPSC-CMs to similar levels as that observed with full length FHF1_{A}, while transduction of GFP as a negative control resulted in no change (FIGS. 5I-5K). Furthermore, FixR had no effect on HD iPSC-CMs (FIGS. 5K, 13L and 13M). In all, these findings highlight the potential utility of FixR as a therapeutic strategy.

### Acute Inhibition of I_{Na,L} by Cell Permeable FixR

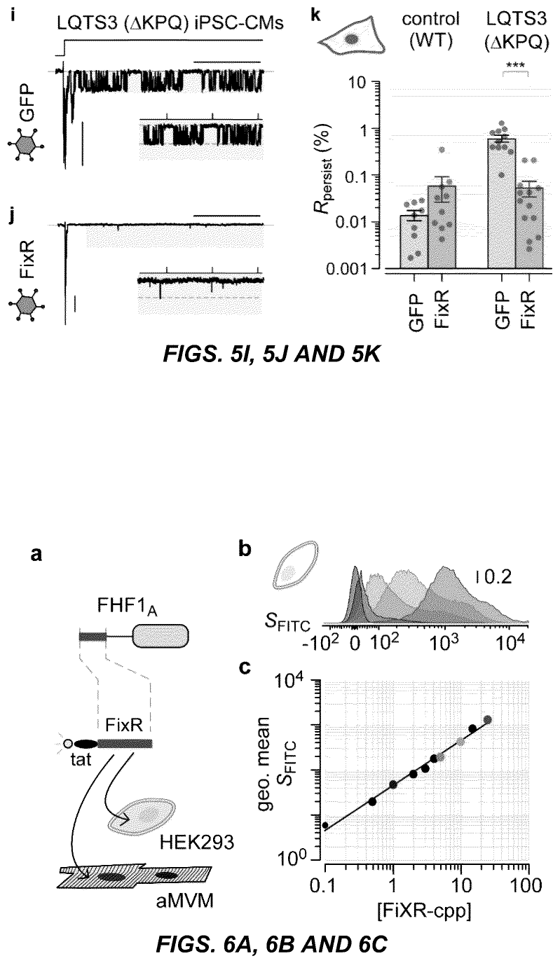

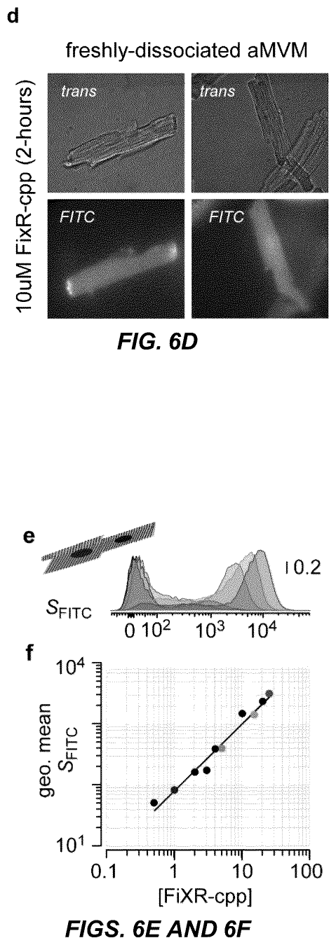

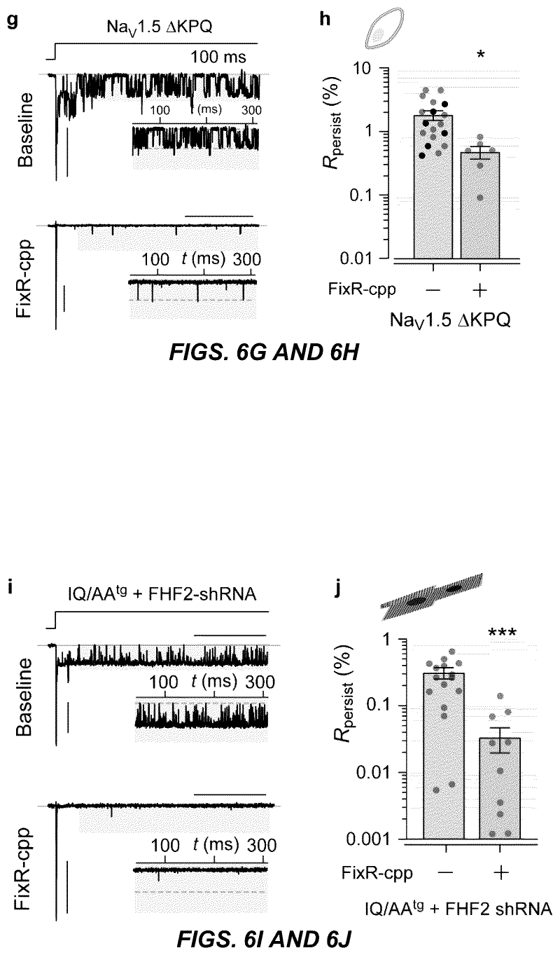

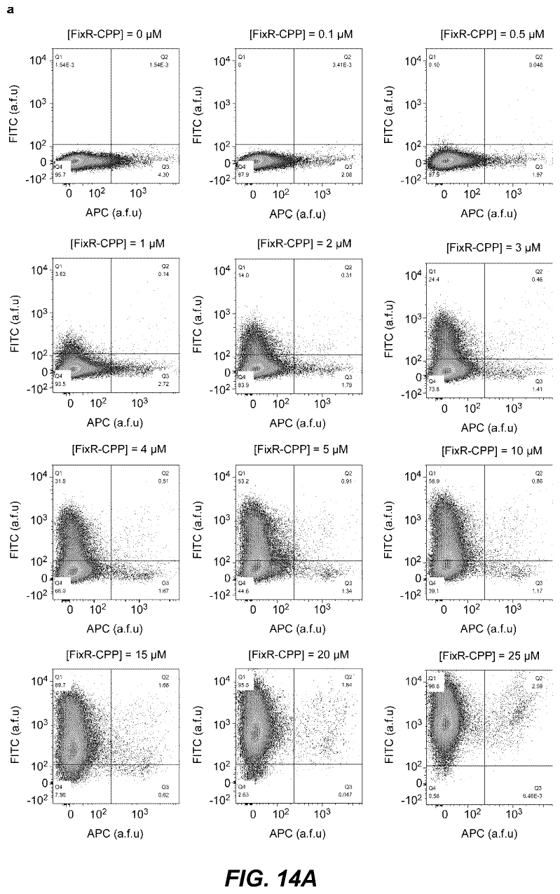

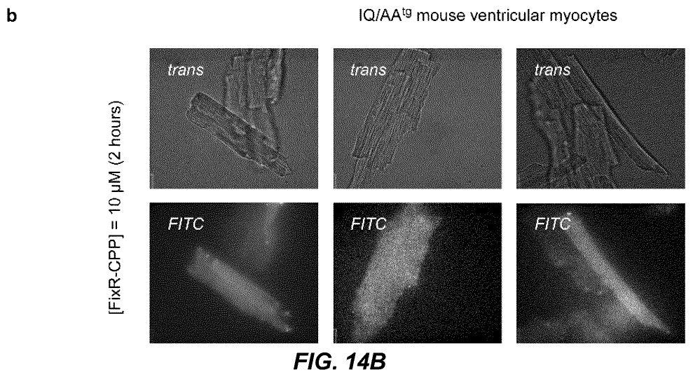

A key hurdle in the practical use of FixR as an I_{Na,L} inhibitor is that it acts by interacting with channel cytosolic domain, and therefore requires intracellular delivery for robust function. Various cell penetrating peptides have been used to facilitate cellular uptake of small proteins, fluorescent markers, siRNA, and nanoparticles. We reasoned that fusing a protein transduction domain from HIV-1 trans-activators of transcription (TAT) to FixR (FixR-cpp, FIG. 6A) may permit intracellular delivery of the peptide. To examine this, we synthesized FixR-cpp with an amino-terminal FITC tag to fluorescently monitor cellular entry (FIG. 6A). We incubated HEK293 cells with varying concentrations of FixR-cpp for 2 hours and measured fluorescence intensity from individual cells using flow-cytometry. We found a concentration-dependent increase in the uptake of FixR-cpp with 10 μM concentration yielding an ˜100-fold enhancement in fluorescence intensity (FIGS. 6B, 6C and 14A). To probe the functional effect of FixR-cpp, multichannel recordings were performed on HEK293 cells transfected with Na_{V}1.5 ΔKPQ mutant channel following a 2-hour incubation with 10 μM FixR-cpp. In comparison to the control conditions, FixR-cpp incubation diminished I_{Na,L} (FIGS. 6G and 6H).

Encouraged by these effects in heterologous cells, we probed the effect of FixR-cpp on I_{Na,L} in cardiomyocytes. Epifluorescence imaging confirmed robust uptake of FixR-cpp into freshly dissociated cardiomyocytes as evident from increased cytosolic yellow fluorescence (FIG. 6D and 14B). The dose-dependence of peptide uptake into aMVM was probed by incubating freshly isolated aMVM with varying concentrations of FixR-cpp for 2 hours and by measuring fluorescence intensity using a flow-cytometer. We found a concentration-dependent increase in the uptake of FixR-cpp with 10 μM concentration yielding an ˜150-fold enhancement in fluorescence intensity (FIGS. 6E and 6F). To probe functional changes in I_{Na,L}, we cultured ventricular myocytes from the IQ/AA^{tg} mouse transduced with FHF2-shRNA to downregulate endogenous protective FHF. As in FIG. 1E, this maneuver results in increased I_{Na,L} (FIGS. 6I and 6J). Incubation of FixR-cpp resulted in an ˜10-fold reduction in Rₚₑᵣₛᵢₛₜ (FIGS. 6I and 6J). These findings confirm the functionality of FixR-cpp and suggests its potential utility as a powerful mechanism-inspired strategy to inhibit I_{Na,L} in cardiomyocytes.

Increased late Na current has emerged as a dominant contributor to diverse cardiac pathologies, and is therefore, a highly sought-after target for antiarrhythmic therapeutics. Although previous studies have shown that intracellular FHF can tune I_{Na,L}, the precise contribution of this modulation to cardiac physiology and pathophysiology, as well as the underlying molecular mechanisms are yet to be fully determined. Beyond this, the full potential of this modulatory scheme for engineering selective I_{Na,L} inhibitors remains untapped. By leveraging multiple synergistic model systems and in-depth biophysical analysis, this study furnishes three key advances. First, systematic analysis of FHF1-3 variants revealed an unexpected bidirectional modulation of I_{Na,L} amplitude depending on the specific FHF isoform/splice-variant. Second, adenoviral manipulation of FHF levels in both cultured adult mouse ventricular myocytes and LQTS3 patient-derived iPSC-cardiomyocytes unambiguously establish the physiological importance of FHF regulation of I_{Na,L}, and highlights its potential role in switching disease pathogenic mechanisms. Third, through structural reductionism, we demonstrate that a minimal FHF domain may be repurposed as a potent I_{Na,L} inhibitor and provides a template for developing a new class of antiarrhythmics.

The four FHF isoforms (FHF1-4) have emerged as versatile Na_{V} modulators in neurons and cardiomyocytes. Structurally, the primary FHF binding site resides in the CTD, in close proximity to the CaM binding IQ domain. FHFs regulate multiple facets of channel function including shifts in voltage-dependence of fast inactivation, trigger “long-term inactivation,” promote Na_{V} trafficking, suppress Ca²⁺/CaM-dependent inhibition, and inhibit I_{Na,L}.

For I_{Na,L} regulation, which is of high relevance pathophysiologically, the prevailing view is that the binding of the FHF core-domain with the CTD allosterically alters the stability of the III-IV linker to prevent late channel openings, in essence mimicking the action of CaM. As such, FHF regulation of I_{Na,L} has been presumed to be limited to mutations in the CTD that alter CaM interaction. Our present findings reveal a more nuanced scheme of channel modulation. First, we find that FHF inhibition of I_{Na,L} is isoform/splice-variant specific. That is, only certain “protective” FHF variants containing an amino-terminal domain inhibit I_{Na,L} while others either upregulate I_{Na,L}, or yield no change. Thus, the net magnitude of I_{Na,L} can be continuously adjusted in the manner of a rheostat, depending on the relative abundance of distinct FHF isoforms/variants in the cell. Second, we find that the “protective” FHF variants arc surprisingly general in inhibiting I_{Na,L} triggered by various mechanisms, including channelopathic mutations in segments outside of the CaM binding domain, as well as from channel phosphorylation. Third, functional regulation of I_{Na,L} by FHF does not depend on its interaction with the carboxy-tail binding domain, as deletion of a large swath of the CTD (E[1810]*) preserved robust I_{Na,L} inhibition. Fourth, in depth analysis of FHF domains responsible for I_{Na,L} inhibition revealed that the β-trefoil core domain is dispensable; instead, a sub-segment within the alternatively spliced FHF amino-terminus suffices to confer I_{Na,L} regulation. Although this region overlaps with the previously identified long-term inactivation particle, additional neighboring residues are required for I_{Na,L} regulation. Fifth, FHF was able to enhance channel inactivation when conventional fast inactivation was disabled, by either mutating the “IFM” inactivation particle, or through S6 mutations that occlude accessibility of the transmembrane receptor site for the ‘IFM’ particle. Taken together, our findings are consistent with FHF utilizing an orthogonal mechanism to inhibit Na channels. These findings bear broad ramifications for physiological regulation of late Na current, and for understanding pathogenic mechanisms underlying both inherited and acquired arrhythmias.

Genetic studies have revealed a staggering number of mutations in the SCN5A gene in patients with electrocardiogramormalities and life-threatening cardiac diseases. The associated cardiac syndromes, however, show phenotypic variability and reduced disease expressivity for reasons that are not fully understood. This is particularly evident for mutations in the channel CTD whereby some patients present with a gain-of-function LQTS3 phenotype while others exhibit a BrS phenotype consistent with a loss-of-function effect. The traditional view is that multiple biophysical defects can trigger distinct phenotypes in an idiosyncratic manner. Our recent analysis of Na_{V}1.5 CT channelopathic mutations that disrupted CaM binding showed two distinct effects, specifically a reduction in peak P_{O} and a concomitant increase in I_{Na,L}. Although these differences appear to be coarsely consistent with LQTS3 and BrS phenotypes, respectively, in silico modeling suggested that these changes alone are insufficient and additional factors were required to switch arrhythmogenic mechanisms. Our present findings suggest that endogenous FHF in cardiomyocytes may subserve this role. In particular, human ventricular myocytes endogenously express multiple FHF isoforms/splice-variants including the “non-protective” FHF1_{B} and certain “protective” FHF2 variants, albeit at lower levels. One hypothesis is that the relative proportion of “protective” versus “non-protective” variants may fluctuate in different individuals. Such variability may in turn result in differential manifestation of I_{Na,L} at the cellular level, ultimately contributing to reduced disease expressivity. Future studies that correlate expression levels of specific FHF splice-variants/isoforms in different individuals with their cardiac phenotype may help discern this possibility.

Physiologically, I_{Na,L} plays a significant role in shaping the plateau phase of the AP, and for ion homeostasis. The basal level of I_{Na,L} is partially determined by CaMKII phosphorylation of Na_{V}1.5. β-adrenergic stimulation further upregulates I_{Na,L} by increased Na_{V}1.5 phosphorylation via both the PKA and the CaMKII pathways. During cardiac pathologies including myocardial ischemia and heart failure, these pathways are chronically upregulated resulting in increased I_{Na,L} that promotes vulnerability for arrhythmias. Our work suggests that this overarching process is finely tuned by endogenous FHFs in cardiomyocytes, potentially furnishing an important feedback mechanism. Interestingly, FHF2 gene expression is upregulated in pathological cardiac hypertrophy in mice and may serve as a compensatory mechanism to counteract the increase in I_{Na,L}. A corollary is that reduced FHF2 regulation may be pathogenic by increasing the likelihood for arrhythmias.

Given its broad relevance to the pathophysiology of acquired and inherited arrhythmias, selective blockade of I_{Na,L} has garnered considerable interest both for physiological studies and as a therapeutic strategy. To date, the dominant strategy for I_{Na,L} inhibition are small-molecule modulators such as ranolazine, GS967, and eleclazine, that target a common transmembrane local-anesthetic binding site. Our results point to FixR as a new template for designing novel I_{Na,L} inhibitors. Compared to ranolazine, FixR exerts a near-complete reversal of I_{Na,L} triggered by either channelopathic mutations or by channel phosphorylation, thus confirming both its generality and potency. Furthermore, robust intracellular delivery of FixR may be attained through either viral gene delivery or through acute application of a synthetic peptide attached to a cell-penetrating moiety. As FixR is genetically encodable, it can be targeted using cell-type specific promotors or could be localized to subcellular domains such as the cardiac dyad or the intercalated disc, potentially permitting unprecedented insights into both the molecular and cell physiological consequences of I_{Na,L}. From a therapeutic perspective, given its relatively small size, peptidomimetics that structurally resemble FixR may furnish an alternate strategy for developing a novel class of small molecule I_{Na,L} inhibitors.

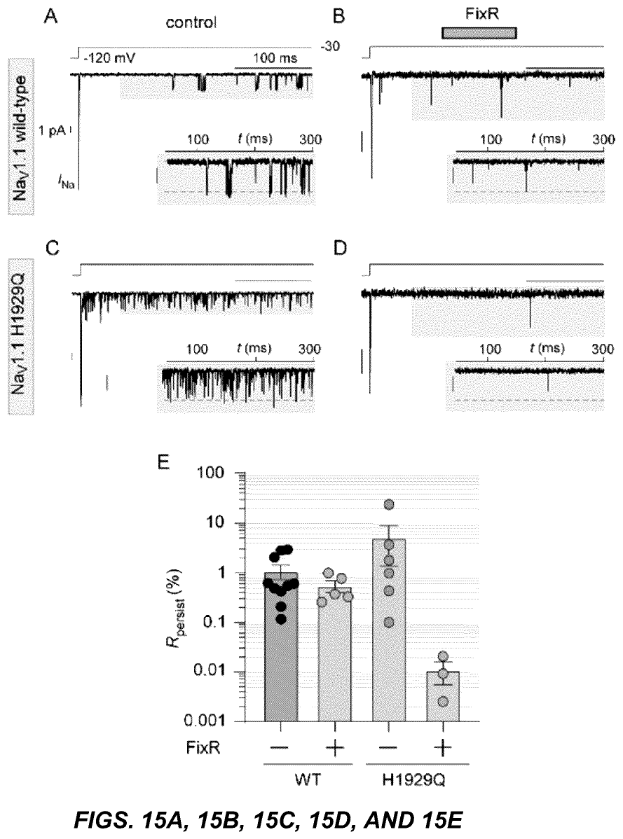

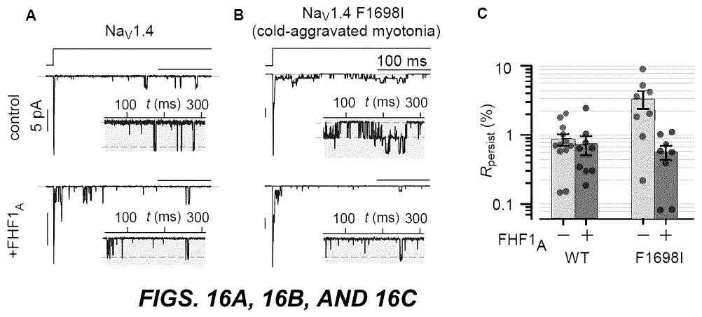

Of broader relevance, increased I_{Na,L} in neurons and in skeletal muscle are linked to epilepsy or neurodevelopmental delay, and myotonia, respectively. As various FHF isoforms/splice-variants are differentially expressed in these tissues, the general framework for FHF modulation developed here may lend new insights in complex pathophysiological mechanisms. To demonstrate this, we considered the NaV1.1 channel which is important for neuronal action potential generation. Channelopathic mutations in NaV1.1 are linked to epilepsy and neurodevelopmental delay. These mutations disrupt NaV1.1 inactivation resulting in increased late Na current. This alteration can lead to an imbalance in the excitatory versus inhibitory neurons resulting in broad changes in brain function. Here we considered H1929Q mutation that was identified in a patient with Early infantile epileptic encephalopathy with suppression bursts. We found that this mutation increases late Na current (FIGS. 15A-B,E). Consistent with our results with Na_{V}1.5 mutations, we found that FixR reduces late Na current for the channelopathic mutation but has no effect on wild-type Na_{V}1.1 channels (FIGS. 15 C-D,E). From a therapeutic perspective, a key advantage of FixR is that it is genetically encodable and therefore can be targeted specifically to excitatory versus inhibitory neurons. This targeted inhibition of late Na current may allow for restoration of excitation-inhibition balance that is essential for brain function. In addition, we also considered Na_{V}1.4 channels which are expressed predominantly in the skeletal muscle. Defective Na_{V}1.4 inactivation and increased late Na current is linked to various forms of myotonia. Here, we considered the F16981 mutation that is linked to cold-aggravated myotonia. As expected, this mutation strongly increased I_{Na,L} which may contribute to increased cellular excitability that contributes to myotonia (FIG. 16A-C). Co-expression of FHF1_{A} with wild-type Na_{V}1.4 resulted in minimal change in I_{Na,L} (FIG. 16A,C). However, FHF1_{A} yielded a strong reduction in I_{Na,L} of Na_{V}1.4 F1698I mutant, similar levels as wild-type channels (FIG. 16 B-C). Thus, the broader FHF regulatory mechanism may also be highly relevant for promoting proper skeletal muscle function. In all, FixR may provide a convenient strategy to dissect the functional importance of I_{Na,L} in these settings with high precision.

### Molecular Biology

The human Na_{V}1.5 channel corresponds to clone M77235.1 (GenBank) and is subcloned into pGW 1 vector with HindIII and SaII. To facilitate construction of channelopathic mutations in the channel carboxy-terminus, we introduced silent mutations into Na_{V}1.5 to introduce an NruI cutting site near the channel carboxy-terminus (5329-gtggccacg-5337 into 5329-gtCgcGacg-5337). Subsequently, we used overlap-extension PCR and ligated the PCR product into NaV1.5/pGW1 using NruI and XbaI restriction sites (mutations: E1784K, S1904L, IQ/AA, and Q1909R). For generating the ΔKPQ and IFM/IQM mutations, we used overlap-extension PCR and ligated in Na_{V}1.5/pGW1 following restriction digest using KpnI and XbaI site. For generation of Na_{V}1.5 truncations, we PCR amplified relevant segments using a forward primer upstream of the KpnI site and a reverse primer that truncated at the appropriate location (Δ1810 and Δ1885) and included an XbaI site and ligated into Na_{V}1.5/pW1 vector. For LILA/WICW mutations, we used overlap extension and used restriction site AgeI and NheI for subsequent ligation. All segments subjected to PCR amplification were verified by sequencing. For generating human FHF2 splice-variants, we synthesized gene fragments and ligated into pcDNA3 using BamHI/EcoRI restriction sites. Human FHF1A and FHF1B were subcloned into ECFP-N3 vector, FHF2 splice variants and FHF3 were subcloned into pcDNA3. Truncations of the FHF1A amino-terminus (NT) were generated as fusion proteins with Venus fluorescent protein in the carboxy-terminus by encoding the relevant segments as noted in FIG. 12A in the forward PCR primer and the fluorophore as the template. Following PCR amplification, each fragment was ligated into pcDNA3 following restriction digest using NheI and XhoI enzymes. Plasmid encoding PKA catalytic subunit was constructed from pDONR222-PRKACA as described previously.

### Adenovirus Generation

Adenovirus encoding FHF1A-P2A-Venus, FixR-P2A-Venus, FHF2-shRNA, scrambled-shRNA, and eGFP were obtained from Vector Biolabs. Briefly, FHF1A-P2A-Venus and FixR-P2A-Venus, were synthesized as gene-fragments (Twist Biosciences) and were flanked by BamHI and EcoRI restriction sites. The sequence of FHF1A segment encoding for FixR is: 5′-ATGGCTGCGGCGATAGCCAGCTCCTTGATCCGGCAGAAGCGGCAGGCG AGGGAGTCCAACAGCGACCGAGTGTCGGCCTCCAAGCGCCGCTCCAGCCCCAGC AAAGACGGGCGCTCC-3′ (SEQ ID NO. 1). Subsequently, the gene fragments were subcloned into the dual CCM+ vector containing a CMV promoter using BamHI and EcoRI restriction sites. Human Type 5 (dE1/E3) adenovirus were packaged and purified to >1.0×10¹⁰ PFU/mL. FHF2-shRNA adenovirus were constructed with the following sequence: 5′-CACCGCACTTACACTCTGTTTAACCCTCGAGGGTTAAACAGAGTGTAAGTGCTTT TT-3′ (SEQ ID NO. 3) driven by a U6 promoter. Of note, the shRNA targets both murine and human FG13/FHF2 given the sequence identity in this region. The scrambled-shRNA control (Cat #: 1122) and GFP (Cat #: 1060) was also purchased from Vector Biolabs.

### Cell Culture and Transfection

HEK293 cells (ATCC CRL1573) were cultured on glass coverslips in 60-mm dishes and transfected using the Ca²⁺-phosphate method as previously described. For electrophysiology experiments, we co-transfected 4-8 μg of cDNA encoding the desired channel variant with 4 μg of YFP, and 1 μg of simian virus 40 T-antigen. For experiments evaluating effect of FHF, we transfected the relevant FHF variant at 1:1 ratio as the α-subunit. For experiments considering competition between FHF1/2 isoforms, we co-transfected FHF1_{B} and FHF2_{S} variants at specified ratios (ranging from 1:1 to 1:10). The culture media was replaced following 4 h of transfection. Electrophysiology recordings were then performed at room temperature 1-2 days following transfection.

### Multi-Channel Analysis of Late Na⁺ Current

Multichannel records were obtained in the on-cell configuration with either HEK293 cells or in aMVM as in our previous study. The pipette contained (in mM): 140 NaCl; 10 HEPES; 0.5 CaCl₂; at 300 mOsm, adjusted with tetraethylammonium methanesulfonate; and pH 7.4 adjusted with tetraethylammonium hydroxide. To zero membrane potential, the bath contained (in mM): 132 K⁺-glutamate; 5 KCl; 5 NaCl; 3 MgCl; 2 EGTA; 10 glucose; 20HEPES; at 300 mOsm adjusted with glucose; and pH 7.4 adjusted with NaOH. Data were acquired at room temperature using the integrating mode of an Axopatch 200A amplifier (Axon Instruments, Molecular Devices). Patch pipettes (3-10 MΩ) were pulled from ultra-thick-walled borosilicate glass (BF200-116-10; Sutter Instruments) using horizontal puller (P-97, Sutter Instruments), fire polished with a microforge (Narishige), and coated with Sylgard™ brand silicone elastomer (Dow Corning). Elementary currents were low-pass filtered at 2 kHz with a four-pole Bessel filter and digitized at 200 kHz with an ITC-18 unit (Instrutech), controlled by custom MATLAB software (Mathworks). For each pulse, we obtained P/8 leak pulses. Leak subtraction was performed using an automated algorithm which fit the kinetics of the leak current or the capacitive transient with convex optimization with L1 regularization. Following leak subtraction, the unitary current for each patch was estimated using an amplitude histogram. Each stochastic trace was subsequently idealized. The ensemble average from 50-100 stochastic traces was computed for each patch and normalized to the peak current. The average late current for each patch (Rₚₑᵣₛᵢₛₜ) was computed as the average normalized P_{O} following 50 ms of depolarization.

### Statistical Analysis

All statistical analysis was performed using Graphpad Prism 9.1.1. Data were tested using D′Agostino-Pearson normality test. For normally distributed data requiring multiple comparisons, we used one-way ANOVA followed by Dunnett's multiple comparisons. For nonnormally distributed data requiring multiple comparisons, a Kruskal-Wallis test followed by a Dunn's post hoc test were performed. For comparisons between two groups, 2-tailed Student's t test was used for normally distributed data, and a Mann-Whitney U test was used for nonnormally distributed data. Differences were considered statistically significant at values of P<0.05.

### Whole-Cell Recordings

Whole-cell recordings were obtained at room temperature with an Axopatch 200B amplifier (Axon Instruments). Electrodes were made of borosilicate glass (World Precision Instruments, MTW 150-F4), yielding pipets of 1-2 MΩ resistance, which was compensated by >70%. Pipets were fabricated with a horizontal micropipette puller (model P-97, Sutter Instruments) and fire polished with a Microforge (Narishige). Data acquisition utilized an ITC-18 (Instrutech) data acquisition unit controlled by custom MATLAB software (Mathworks). Currents were low-pass filtered at 2 kHz before digitization at several times that frequency. P/8 leak subtraction was used. Cells were maintained at a holding potential of −120 mV.

### Mouse Models

The Institutional Animal Care and Use Committee at Columbia University approved all animal experiments. The pWT and IQ/AA lines were generated as described previously. In brief, the human heart Na⁺ channel α-subunit cDNA (hH1; Na_{V}1.5) was fused to a vector containing the modified murine α-MHC, tetracycline-inducible promoter. The Na_{V}1.5 channel was engineered to be TTX-sensitive by inserting a C374Y mutation. A 3X-FLAG epitope was ligated in-frame to the N-terminus. These mice, in a B6CBA/F2 hybrid background, were bred with cardiac-specific rtTA mice in a FVB/N background, obtained via MMRRC, to generate doxycycline-inducible transgenic mice. Both male and female mice were used in all experiments. Male and female mice, 6-weeks to 4-months of age were used. Gender had no effect on the outcomes of any experiment. Number of animals were at least 3 per genotype.

### Isolation, Culture, and Adenoviral Transduction of aMVM

Mice ventricular myocytes were isolated by enzymatic digestion using a Langendorff perfusion apparatus as previously described. Cardiomyocytes were isolated from 8- to 12-week-old non-transgenic and transgenic mice. After isolation, the cells were resuspended in perfusion solution with fetal bovine serum (FBS, 5%) and calcium was added gradually to a final concentration of 0.5 mM. Cells were plated on laminin (Corning) coated glass coverslips initially in “plating” media composed of MEM medium with Earle's salts and L-glutamine (Gibco) plus 5% FBS, 1% penicillin/streptomycin, and 10 mM 2,3-butanedione monoxime (BDM). Cells were allowed to adhere for 2 hour before switching to “maintenance” media containing MEM with Earle's salts, L-glutamine, 1% penicillin-streptomycin, bovine serum albumin (0.5 mg/ml), 10 mM BDM, 1% insulin-transferrin-selenium (Gibco), 5 mM creatine, 5 mM taurine, 2 mM L-carnitine, 25 μM Blebbistatin. For manipulation of FHF levels, 1-2 μL of relevant adenovirus was resuspended in culture medium and added to cells. The “maintenance” media was replaced 24 h to reduce potential toxicity from adenovirus. The efficacy of viral transduction was assessed 24-48 hours post infection.

### Generation of FixR-cpp and Quantification of Uptake into HEK293 Cells and aMVM

The peptide encoding FixR-cpp was chemically synthesized by Genscript (98% purity) to contain the cell permeating viral TAT sequence in the amino-terminus: YGRKKRRQRRRAAAIASSLIRQKRQARESNSERVSASKRRSSPSKG (SEQ ID NO. 2). The amino terminus of the peptide contained a FITC fluorophore to visualize cell uptake. The peptide was resuspended in DMSO. Flow cytometric assays were performed to quantify FixR cellular uptake in both HEK293 cells and freshly dissociated aMVM. HEK293 cells were cultured in 12 well plates and incubated with different concentrations of FixR-cpp for 2 hours and stained for cell death detection using LIVE/DEAD™ Fixable Far Red Dead Cell Stain Kit (ThermoFisher Scientific, L10120). Similarly, non-TG and IQ/AA^{tg} freshly dissociated aMVM were incubated with various concentrations of FixR-cpp for 2 hours in Tyrode's solution (138 mM NaCl, 4 mM KCl, 1 mM MgCl₂, 10 mM HEPES (pH 7.4 using NaOH), 0.2 mM NaHPO₄ 5 mM D-glucose, 1 mM CaCl₂). For cellular uptake measurements, we utilized an LSR II (BD Biosciences) flow cytometer equipped with 405 nm, 488 nm, and 633 nm lasers for excitation and 18 different emission channels. Forward and side scatter signals were detected and used to gate for single and healthy cells. Flow cytometric signals were collected at medium flow rate (2k-8k events/sec). Histograms from the Fluorescein 5(6)-isothiocyanate (FITC) and Allophycocyanin (APC) channels for the single-cell population were obtained and analyzed using FlowJo (version X, FlowJo LLC).

### Computational Ventricular Action Potential Modeling

Computational modeling to predict the effect of competition between FHF1/2 splice-variants/isoforms on ventricular action potentials were performed with the ToR-ORd model simulated using MATLAB. We tuned the amplitude of late Na current based on the empirical relationship between Rₚₑᵣₛᵢₛₜ and the ratio of FHF1_{B} to FHF2_{S} in FIG. 2O. Specifically, for the IQ/AA mutant, the late Na current was modified by the following multiplier: G_{Na,L}=(7.254−7.0989/(1+(ρ/0.4279)^{3.340}))/0.17. Since, the level of I_{Na,L} for Na_{V}1.5 wild-type channels is similar in the presence of FHF1_{B} or FHF2_{S}, we modified the above equation to be G_{Na,L}=(0.2−0.0449/(1+(ρ/0.4279)^{3.340}))/0.17. Action potentials were simulated for 100 beats at various heart rates and only the final two beats were considered for measurement of APD.

### Differentiation of iPS-CMs

Differentiation into cardiomyocytes was performed by modifying a previously established protocol. hiPSCs were maintained in mTeSR medium (Stem Cell Technologies) and passaged every 4-6 days onto Matrigel (Corning)-coated plates before differentiation. On day 0 (start of differentiation), hiPSCs were treated with 1 mg/mL Collagenase B (Roche, Cat #11088807001) for 1 hour, or until cells detached from the plates, to generate embryoid bodies (EBs). Cells were then collected and centrifuged at 300 ref for 3 minutes, and resuspended as small clusters of 50-100 cells by gentle pipetting in CM differentiation medium, composed of RPMI 1640 (Thermo Fisher Scientific, Cat #11875085) containing 2 mM/L L-glutamine (Thermo Fisher Scientific, Cat #25030149), 4×10⁻⁴ M monothioglycerol (Millipore Sigma, Cat #M6145), and 50 μg/mL ascorbic acid (Millipore Sigma, Cat #A4403). Differentiation medium was supplemented with 2 ng/mL BMP4 (R&D Systems) and 10 μM Rock inhibitor (Y-27632 dihydrochloride, Tocris Fisher Cat #1254/50), and EBs were cultured in Ultra-Low attachment 6-well plates (Corning Costar, Cat #3471) in a humidified incubator at 37° C. in 5% CO₂, 5% O₂. On day 1, medium was changed to differentiation medium supplemented with 20 ng/mL BMP4 (R&D Systems), 20 ng/ml Activin A (R&D Systems), 5 ng/mL bFGF (R&D Systems). On day 3.5, EBs were harvested and washed once with RPMI 1640. Medium was changed to differentiation medium supplemented with 5 ng/mL VEGF (R&D Systems) and 5 μM XAV939 (Reprocell-Stemgent, Cat #04-0046). From this point on, every 2-3 days medium was replaced with medium supplemented with 5 ng/mL VEGF (R&D Systems) only.

It is to be understood that the treatment and method for inhibiting late Na current is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A treatment composition for inhibiting late Na current, the composition comprising a viral or plasmid vector encoding FixR.

2. The treatment composition according to claim 1, wherein the viral or plasmid vector encoding FixR comprises the sequence set forth in SEQ ID NO: 1.

3. The treatment composition according to claim 1, wherein the FixR comprises the protein sequence set forth in SEQ ID NO: 5.

4. The treatment composition according to claim 1, wherein the FixR comprises the protein sequence set forth in SEQ ID NO: 4.

5. A treatment composition for inhibiting late Na current, the composition comprising a peptide inhibitor of late Na current, wherein the peptide inhibitor is FixR.

6. The treatment composition according to claim 5, wherein FixR is fused to a cell penetrating peptide (FixR-CPP).

7. The treatment composition according to claim 6, wherein FixR-CPP comprises the protein sequence set forth in SEQ ID NO: 2.

8. A method for inhibiting late Na current, comprising the step of administering to a patient in need thereof an effective amount of a treatment composition comprising a viral or plasmid vector encoding FixR or a peptide inhibitor of late Na current, wherein the peptide inhibitor is FixR.

9. The method according to claim 8, wherein the patient is being treated for one or more of the following: a cardiac pathology, a neurological/neuropsychiatric disorder, and a skeletal muscle condition.

10. The method according to claim 8, wherein the treatment composition comprising the viral or plasmid vector is administered, the method comprises administering to a patient in need thereof a treatment composition comprising an effective amount of human adenovirus type 5 genetically modified with the sequence set forth in SEQ ID NO: 1.

11. The method according to claim 8, wherein the treatment composition comprising FixR is administered, the treatment composition comprises FixR fused to a cell penetrating peptide (FixR-CPP), wherein the protein sequence of FixR-CPP is set forth in SEQ ID NO: 2.

12. The method according to claim 8, wherein the patient is being treated for one or more of the following: arrhythmia, epilepsy and autism spectrum disorders, pain-related diseases, and myotonia.

13. The method according to claim 8, wherein the patient is being treated for a cardiac pathology.

14. The method according to claim 13, wherein the patient is being treated for an arrhythmia.

15. The method according to claim 8, wherein the method comprises administering to the patient in need thereof an effective amount of the treatment composition comprising the viral or plasmid vector encoding FixR.

16. The method according to claim 8, wherein method comprises administering to the patient in need thereof an effective amount of the treatment composition comprising FixR.

17. The method according to claim 16, wherein the treatment composition comprising FixR comprises FixR fused to a cell penetrating peptide (FixR-CPP).

18. The method according to claim 16, wherein the FixR comprises at least 35 amino acids residues.

19. The method according to claim 16. wherein the FixR comprises at least 35 amino acids residues from the amino terminus of FHF1A.


SEQUENCE LISTING (6 sequences)

SEQ ID NO: 1 - DNA, 117 bp, Human Adenovirus
  source 1..117: mol_type = genomic DNA; serotype = Type 5
atggctgcggcgatagccagctccttgatccggcagaagcggcaggcgagggagtccaacagcgaccgagtgtcggcctccaagcgccgctccagccccagcaaagacgggcgctcc

SEQ ID NO: 2 - protein, 46 aa, synthetic construct
YGRKKRRQRRRAAAIASSLIRQKRQARESNSERVSASKRRSSPSKG

SEQ ID NO: 3 - DNA, 57 bp, synthetic construct
  source 1..57: mol_type = other DNA
caccgcacttacactctgtttaaccctcgagggttaaacagagtgtaagtgcttttt

SEQ ID NO: 4 - protein, 39 aa, synthetic construct
MAAAIASSLIRQKRQARESNSDRVSASKRRSSPSKDGRS

SEQ ID NO: 5 - protein, 35 aa, synthetic construct
AAAIASSLIRQKRQARESNSERVSASKRRSSPSKG

SEQ ID NO: 6 - protein, 11 aa, synthetic construct
YGRKKRRQRRR